SEQUENCE LISTING (2 sequences)

SEQ ID NO: 1 - protein, 13 aa, Homo sapiens
  MOD_RES (1)..(1): Acetylation
SYSMEHFRWGKPV

SEQ ID NO: 2 - DNA, 19 bp, Artificial
  Tetracycline operator 2
tccctatcagtgatagaga

(19) United States
(12) Patent Application Publication
(10) Pub. No.: US 2009/0081197 A1
(43) Pub. Date: Mar. 26, 2009

(54) Methods for Selection of Melanocortin Receptor-Specific Agents for Treatment of Obesity

(75) Inventors: Kevin D. Burris, Indianapolis, IN (US); Shubh D. Sharma, Cranbury, NJ (US)

Correspondence Address: MORGAN LEWIS & BOCKIUS LLP, 1111 PENNSYLVANIA AVENUE NW, WASHINGTON, DC, 20004, US

(73) Assignee: Palatin Technologies, Inc., Cranbury, NJ (US)

(21) Appl. No.: 12/130,299

(22) Filed: May 30, 2008

Related U.S. Application Data
(60) Provisional application No. 60/924,836, filed on Jun. 1, 2007.

(51) Int. Cl. A61K 39/395; G01N 33/53; A61P 3/00; C12Q 1/68

(52) U.S. Cl. 424/130.1; 435/7.2; 435/6

(57) ABSTRACT

Provided are methods for selecting melanocortin receptor-specific compounds for attenuating food intake and for treatment of specific disease conditions, including treatment of obesity and related energy homeostasis or feeding disorders characterized by excess weight gain, without inducing a sexual response, and methods for selecting compounds for treatment of sexual dysfunction. Further provided are pharmaceutical preparations defined by such methods, and methods and preparations for attenuating food intake and treatment of said conditions.

### CROSS-REFERENCE TO RELATED APPLICATIONS

This application claims priority to U.S. Provisional Patent Application No. 60/924,836 filed Jun. 1, 2007, which is herein incorporated by reference in its entirety.

A related U.S. application entitled Compounds and Methods for Treatment of Obesity is being filed concurrently herewith, Attorney Docket No. 056291-5365, and the specification and claims thereof are incorporated herein by reference.

### BACKGROUND OF THE INVENTION

1. Field of the Invention (Technical Field)

The present invention relates to screening and selection methods for selecting melanocortin receptor-specific compounds for attenuating food intake, such as for the treatment of obesity and related energy homeostasis or feeding disorders characterized by excess weight gain, which compounds do not induce a sexual response, including a penile response, and to particular compounds thus selected. The invention also relates to related screening and selection methods for selecting melanocortin receptor-specific compounds for treating sexual dysfunction, such as male erectile dysfunction.

2. Description of Related Art

Note that the following discussion refers to a number of publications by author(s) and year of publication, and that due to recent publication dates certain publications are not to be considered as prior art vis-à-vis the present invention. Discussion of such publications herein is given for more complete background and is not to be construed as an admission that such publications are prior art for patentability determination purposes.

A family of melanocortin receptor types and subtypes have been identified, including melanocortin 1 receptors (MC1-R) expressed on normal human melanocytes and melanoma cells, melanocortin 2 receptors (MC2-R) for adrenocorticotropin (ACTH) expressed in cells of the adrenal gland, melanocortin 3 and melanocortin 4 receptors (MC3-R and MC4-R), expressed primarily in cells in the hypothalamus, mid-brain and brainstem, and melanocortin 5 receptors (MC5-R), expressed in a wide distribution of tissues.

It has been hypothesized that compounds specific for MC3-R or MC4-R are useful in regulation of energy homeostasis, including use as agents for attenuating food intake and body weight gain, for use in treatment of anorexia and cachexia, for treatment of obesity, and treatment of other food intake and metabolism-related indications. However, the mechanism of action of compounds specific for MC3-R or MC4-R as agents for regulation of energy homeostasis has not been fully elucidated.

Most MC4-R agonist compounds that have been evaluated for use in attenuating food intake and body weight gain also have other systemic effects relating to the melanocortin system, including inducing or facilitating penile erection in males. These effects render most MC4-R agonists unsuitable for use in treatment of obesity. There is a need for melanocortin-specific agents which are efficacious for treatment of obesity, but without other systemic effects, such as induction of penile erections in males, or a sexual response generally in mammals.

It has been shown that melanocortin receptors, including MC4-R, can express a basal or constitutive level (i.e., unstimulated) of adenylyl cyclase activity, even in the absence of a specific agonist. Adenylyl cyclase is an enzyme of the lyase class that catalyzes the formation of 3′,5′-cyclic adenosine monophosphate (cAMP) from ATP (adenosine triphosphate, the 5′ triphosphate of adenosine). It is clear that earlier models of receptor theory are not sufficient to explain observed results. Two papers by T. Kenakin, among others, provide receptor models, including a ternary complex model, specifically for G-protein coupled receptors, which recognize that efficacy of a compound may vary based on receptor density, efficiency of receptor coupling, and other environmental factors relating to the receptor. (Kenakin T. Efficacy at G-Protein-Coupled Receptors (review). Nature Reviews 1:103-110 (2002); Kenakin T. Principles: Receptor theory in pharmacology (review). TRENDS in Pharmacol. Sci. 25:186-192 (2004)).

Most, if not all, MC4-R agonist compounds explored for attenuating food intake also induce a sexual response, including a penile erection response in males, and in addition all or virtually all MC4-R agonist compounds reported for attenuating food intake or body weight gain in animal models have shown a “rebound” effect, with animals gaining weight equal to or, in many instances exceeding, controls upon cessation of administration of the compounds. There is thus a need for compounds which attenuate food intake or body weight gain without causing a rebound effect upon cessation of administration of the compound.

U.S. Pat. No. 5,908,609, by Lee, Huszar and Gu and assigned to Millennium Pharmaceuticals, Inc., discloses a method for identifying compounds that regulate body weight, including contacting a test compound with a cell which expresses a functional MC4-R, and determining whether the compound activates the receptor. It is claimed that compounds that “activate” MC4-R are identified as compounds for inducing weight loss. However, “activation” is defined in terms of simple induction of cAMP expression in a fixed melanocortin receptor density system, and thus this patent simply claims a method of selecting a MC4-R agonist. Similarly, U.S. Pat. No. 6,716,810, by Brennan and Hochgeschwender and assigned to Eleanor Roosevelt Institute and Oklahoma Medical Research Foundation, discloses methods of regulating body weight, such as by administration of α-MSH, an α-MSH analog, or an α-MSH homolog having agonist activity. Neither patent, nor any other methods disclosed elsewhere, address the selection of a melanocortin receptor-specific compound that is effective for modulating feeding behavior or energy homeostasis, but which does not induce other systemic effects, for example, which does not induce a sexual response.

### SUMMARY OF THE INVENTION

In one aspect, the invention provides a method of selecting a compound as a candidate for attenuating food intake, such as for treating obesity and related energy homeostasis or feeding disorders characterized by excess weight gain, which compound at least substantially does not induce or initiate a sexual response, the method comprising the steps of:
providing a low density melanocortin 4 receptor (MC4-R) system;
providing a high density MC4-R system;
determining the functional activity of the compound in the low density MC4-R system, such as by means of an adenylyl cyclase expression assay;
determining the functional activity of the compound in the high density MC4-R system, such as by means of an adenylyl cyclase expression assay;
selecting the compound as a candidate if the compound is functionally inactive in the low MCR-4 density system and is functionally active in the high density MC4-R system.

In a related aspect, the low density MC4-R system is a low density human MC4-R (hMC4-R) system and the high density MC4-R system is a high density hMC4-R system. In one aspect, the low density MC4-R system has a melanocortin receptor density determined by receptor binding saturation that is at least ten times lower that the melanocortin receptor density in the high density MC4-R system, at least fifty times lower than the melanocortin receptor density in the high density MC4-R system or at least one hundred times lower than the melanocortin receptor density in the high density MC4-R system.

In a related aspect, both the low density MC4-R system and the high density MC4-R system comprise mammalian cells transformed with a gene encoding for MC4-R. In one aspect, the gene encoding for MC4-R comprises a gene encoding for hMC4-R. In one aspect, the gene encoding for MC4-R is under the control of a gene expression variable regulatory system, for example, a variably inducible expression system as known in the art. In one aspect, the gene expression variable regulatory system comprises a tetracycline-regulated mammalian expression system. In one aspect the low density MC4-R system employs (receives) at least ten times less of the inducer of expression, such as tetracycline or a related regulator, than does the high density MC4-R system. In one aspect, the low density MC4-R system employs (receives) at least about one hundred times less inducer of expression, such as tetracycline or a related regulator, than does the high density MC4-R system.

In a related aspect, the low density MC4-R system has a receptor density expressed as a Bₘₐₓ receptor binding saturation value using NDP-α-MSH, such as I¹²⁵-labeled NDP-α-MSH, of between approximately 80 and 20 fmol/mg or between approximately 80 and 40 fmol/mg. In a related aspect, the high density MC4-R system has a receptor density expressed as a Bₘₐₓ receptor binding saturation value using NDP-α-MSH, such as 25-labeled NDP-α-MSH, of at least about 4,000 fmol/mg or at least about 6,000 fmol/mg.

In a related aspect, the compound is functionally inactive in the low MCR-4 density system where the compound has no measurable intrinsic activity, or alternatively has an intrinsic activity of less than about 0.1 (10%), based on maximal stimulation of adenylyl cyclase achievable by the compound in the low hMC4-R density system where the maximal stimulation achieved by α-MSH or NDP-α-MSH is designated as an intrinsic activity of 1.0 (100%).

In a related aspect, the compound is functionally active in the high density MC4-R system where the compound has an intrinsic activity of more than about 0.1 (10%), more than about 0.2 (20%), more than about 0.3 (30%), more than about 0.5 (50%), or more than about 0.7 (70%), based on maximal stimulation of adenylyl cyclase achievable by the compound in the high hMC4-R density system where the maximal stimulation achieved by α-MSH or NDP-α-MSH is designated as an intrinsic activity of 1.0 (100%).

In a second aspect, the invention provides a method of selecting a compound for treating sexual dysfunction, the method comprising the steps of:
providing a low density MC4-R system;
providing a high density MC4-R system;
determining the functional activity of the compound in the low density MC4-R system, such as by means of an adenylyl cylase expression assay;
determining the functional activity of the compound in the high density MC4-R system, such as by means of an adenylyl cylase expression assay;
selecting the compound as a candidate for treating sexual dysfunction if the compound is functionally active in both the low MCR-4 density system and high density MC4-R system.

In a related aspect, the method further comprises the steps of determining the functional status of the compound in a transfected or transformed MC4-R system not under the control of a gene expression variable regulatory system and selecting the compound as a candidate compound further requires that the compound is an agonist in the transformed MC4-R system not under the control of a gene expression variable regulatory system. In a related aspect, the compound is an agonist in the transformed MC4-R system where the compound has an intrinsic activity of more than about 0.7 (70%), based on maximal stimulation of adenylyl cyclase achievable by the compound in the transformed hMC4-R system where the maximal stimulation achieved by α-MSH or NDP-α-MSH is designated as an intrinsic activity of 1.0 (100%).

In a third aspect, the invention provides a method of selecting a compound for attenuating food intake and/or for treating obesity and related energy homeostasis or feeding disorders characterized by excess weight gain which compound at least substantially does not induce or initiate a sexual response, the method comprising the steps of:
providing a low density MC4-R expression system;
providing a high density MC4-R expression system;
determining the functional activity of the compound in an adenylyl cyclase expression assay employing the low density MC4-R system;
determining the functional activity of the compound in an adenylyl cyclase expression assay employing the high density MC4-R system;
selecting the compound if the compound is functionally inactive in both the low MCR-4 density system and the high density MC4-R system.

In a related aspect, the invention further comprises the step of providing a competition inhibition assay for competitive binding with α-MSH as to MC4-R and selecting the compound as a candidate or further candidate compound further requires that the compound inhibits at least about 90% of the binding of α-MSH to MC4-R. In another related aspect, the invention further comprises the step of providing an assay for determining the Ki (nM) of the compound as to α-MSH binding to MC4-R and selecting the compound as a candidate or further candidate compound further requires that the compound has a Ki (nM) of less than about 100, a Ki (nM) of less than about 10, or a Ki (nM) of less than about 1.

In a fourth aspect, the invention provides a pharmaceutical composition for treatment of obesity and/or for attenuating food intake and body weight gain generally, without substantially inducing a sexual response, comprising an effective amount of a melanocortin-4 receptor (MC4-R) specific compound which is functionally inactive at a concentration of about 1000 nM or less in an adenylyl cyclase expression assay at a low MC4-R density but which is functionally active at a concentration of about 1000 nM or less in an adenylyl cyclase expression assay at a high MC4-R density, and one or more of carriers, excipients and adjunct ingredients.

In a fifth aspect, the invention provides a pharmaceutical composition for treatment of obesity and/or for attenuating food intake and body weight gain generally, without substantially inducing a sexual response, comprising an effective amount of a melanocortin-4 receptor (MC4-R) specific compound which is functionally inactive at a concentration of about 1000 nM or less in an adenylyl cyclase expression assay at a low MC4-R density and at a high MC4-R density, and one or more of carriers, excipients and adjunct ingredients.

In a sixth aspect, the invention provides a pharmaceutical composition for treatment of sexual dysfunction, comprising an effective amount of a melanocortin-4 receptor (MC4-R) specific compound which is functionally active at a concentration of about 1000 nM or less in an adenylyl cyclase expression assay at both a low MC4-R density and at a high MC4-R density, and one or more of carriers, excipients and adjunct ingredients.

One embodiment of the present invention provides melanocortin receptor-specific agents useful in treatment of obesity and/or for attenuating food intake and body weight gain generally, the agents characterized in part in that they doenot induce a sexual response in a mammal, including not inducing a penile erection response in a male.

Another embodiment of the present invention provides a method for selection of a melanocortin receptor-specific agent useful in treatment of obesity and/or for attenuating food intake and body weight gain generally, the method characterized in part in that agents are selected which have low or no efficacy with respect to cAMP production in a low density melanocortin receptor expression system, but which have significant efficacy with respect to cAMP production in a high density melanocortin receptor expression system.

Another embodiment of the present invention provides melanocortin receptor-specific agents useful in treatment of obesity and/or for attenuating food intake and body weight gain generally, the agents characterized in part in that they have efficacy, such as by cAMP production, equal to or greater than NDP-α-MSH in a high density melanocortin receptor expression system, but less than NDP-α-MSH, and preferably substantially less than NDP-α-MSH, in a low density melanocortin receptor expression system.

Another embodiment of the present invention provides a method for selection of melanocortin receptor-specific agents useful in treatment of obesity and/or for attenuating food intake and body weight gain generally, the method characterized in part in that agents are selected which have low or no efficacy with respect to cAMP production in a low density hMC4-R (human melanocortin 4 receptor) expression system, but which have significant efficacy with respect to cAMP production in a high density hMC4-R expression system.

Another embodiment of the present invention provides a method for selection of a melanocortin receptor-specific agent useful in treatment of obesity and/or for attenuating food intake and body weight gain generally, the method characterized in part in that agents are selected which have low or no efficacy with respect to cAMP production in a low density melanocortin receptor expression system and in a high density melanocortin receptor expression system.

Another embodiment of the present invention provides a melanocortin receptor-specific agent useful in treatment of obesity and/or for attenuating food intake and body weight gain generally, the agent characterized in part in that it is efficacious for treatment of obesity and/or for attenuating food intake and body weight gain generally without substantially inducing a sexual response, such as without substantially inducing a penile erection response in a male.

Yet another embodiment of the present invention provides a melanocortin receptor-specific agent useful in treatment of obesity and/or for attenuating food intake and body weight gain generally that binds to MC4-R with high affinity but has low intrinsic activity in terms of cAMP expression in a low or moderate MC4-R density expression system.

Yet another embodiment of the present invention is to provide a method of treating obesity and/or for attenuating food intake and body weight gain generally comprising administration of a melanocortin receptor-specific agent that binds to MC4-R with high affinity, preferably with a Ki (nM) at MC4-R of about 15 or less, but has low intrinsic activity in terms of cAMP expression in a low, moderate or high density MC4-R expression system, and is efficacious in treating obesity and/or in attenuating food intake and/or body weight gain.

Yet another embodiment of the present invention provides a method of treating obesity and/or for attenuating food intake and body weight gain generally comprising administration of a melanocortin receptor-specific agent that has a Ki (nM) at MC4-R, determined with respect to NDP-α-MSH, that is half or less than half, and preferably substantially less than half, of the EC₅₀ (nM) for cAMP accumulation at MC4-R in a low or moderate MC4-R density expression system.

Yet another embodiment of the present invention provides a method of treating obesity and/or for attenuating food intake and body weight gain generally comprising administration of a melanocortin receptor-specific agent wherein there is at least no substantial rebound effect, such as no significant immediate rebound effect, or at least no substantial increase in body weight, following cessation of administration of the agent. In this embodiment, the agent at least substantially does not cause an immediate rebound effect, or stated differently, the agent is not associated with any substantial rebound effect, particularly no substantial rebound effect within the first month, or alternatively within the first two months, following cessation of administration.

Yet another embodiment of the present invention provides a method for selection of a melanocortin receptor-specific agent useful in treatment of sexual dysfunction, including male erectile dysfunction and/or female sexual dysfunction, the method characterized in part in that an agent is selected which has significant efficacy with respect to cAMP production in a low hMC4-R density expression system and in a high hMC4-R density expression system.

One aspect of this invention provide compounds that bind to a receptor binding site of α-MSH, such as on MC4-R, preferably human MC4-R, with high affinity yet without causing any activation, or alternatively causing low activation, of adenylyl cyclase activity in a low density melanocortin receptor expression system, but which cause activation of adenylyl cyclase in a high density melanocortin receptor expression system. In another aspect, the compounds are either inactive or are neutral antagonists with respect to α-MSH in low density melanocortin receptor expression system cAMP stimulation assays. In another aspect, the compounds are partial agonists in low density melanocortin receptor systems, but are full agonists in high density melanocortin receptor systems.

In another embodiment, the methods of this invention provide compounds that bind to a receptor binding site of α-MSH, such as on MC4-R, preferably hMC4-R, with high affinity and further cause activation of adenylyl cyclase activity in both a low density melanocortin receptor expression system and a high density melanocortin receptor expression system, which compounds may be employed for treatment of sexual dysfunction, including erectile dysfunction in males and female sexual dysfunction.

Other objects, advantages and novel features, and further scope of applicability of the present invention will be set forth in part in the detailed description to follow, and in part will become apparent to those skilled in the art upon examination of the following, or may be learned by practice of the invention. The objects and advantages of the invention may be realized and attained by means of the instrumentalities and combinations particularly pointed out in the appended claims.

### DETAILED DESCRIPTION OF THE INVENTION

In one broad aspect, the invention describes and discloses methods for the identification and selection of compounds with utility for treatment of obesity and related energy homeostasis diseases, conditions and syndromes, but which compounds do not induce a sexual response in mammals, including a penile erection response in males. In a related aspect, the invention describes and discloses the use of a variety of specified melanocortin receptor-specific compounds selected by such methods that may be employed in the treatment of obesity and related energy homeostasis diseases, conditions and syndromes.

In one embodiment the invention employs an inducible gene expression system to transform cells, such as for example HEK-293 cells or CHO cells, with varying receptor densities of hMC4-R. As used herein, a “transformed” cell system is one constitutively expressing an MC4-R transgene, most preferably where the system is stably transformed for constitutive expression of MC4-R. While preferably the transgene is incorporated into the cell's genome, it is also possible and contemplated that transfection, including stable transfection, may be employed. In one inducible gene expression system using doxycycline as the inducer of gene expression, the receptor density varies from very low, such as for example about approximately 70 fmol/mg, to very high, such as for example about approximately 6300 fmol/mg. By contrast, “normal” transformed or transfected cells, such as those not employing an inducible gene expression system, have a receptor density of approximately about 400 fmol/mg. The invention, in part, relates to the discovery that MC4-R specific compounds with no efficacy in terms of cAMP expression in a very low density system, but that have efficacy (such as equal to or greater than NDP-α-MSH) in terms of cAMP expression in a very high density system, are likely to be effective for treatment of obesity and/or for attenuating food intake and body weight gain generally without inducing a sexual response in a mammal, including a penile response in a male mammal. Conversely, MC4-R specific compounds with efficacy in terms of cAMP expression in both a very low density system and a very high density system, are likely to be effective for treatment of sexual dysfunction, include male erectile dysfunction and female sexual disorder. Without wishing to be bound by theory, it is one hypothesis of the inventors that hMC4-R in very low density expressing cells have a different conformation than hMC4-R in very high density expressing cells. Thus, by way of hypothesis only, G-protein activation may be lower in low density cells unless the ligand induces a change in the conformation of the receptors; alternatively, and again by way of hypothesis only, very low density hMC4-R may be in one or more inactive receptor states, while high or very high density hMC4-R are in one or more active receptor states. It is further hypothesized, again without wishing to be bound by theory, that the observed difference may be due to one or more factors such as receptor internalization, desensitization, oligomerization, phosphorylation or association with other membrane proteins.

In the practice of a method of this invention, a melanocortin receptor-specific agent, and preferably an agent specific for MC4-R, is selected, which agent is characterized in that: (a) the agent attenuates the binding of alphα-melanocyte stimulating hormone (α-MSH) to melanocortin receptors; (b) the agent has a low efficacy, such that the agent is a weak agonist, weak antagonist, neutral antagonist, inverse agonist or protean agonist at MC4-R, and preferably has an efficacy or intrinsic activity in terms of cAMP expression of less than about 50%, more preferably less than about 30%, and most preferably less than about 10% of that of α-MSH in a low density MC4-R expression system; and, (c) optionally and preferably, the agent is further characterized in that the agent has an efficacy in terms of cAMP expression of greater than about 50%, more preferably greater than about 80%, and most preferably equal to or greater than about 100% of that of α-MSH in a high density MC4-R expression system and an efficacy or intrinsic activity in terms of cAMP expression of less than about 50%, more preferably less than about 30%, and most preferably less than about 10% of that of α-MSH in a low density MC4-R expression system.

The melanocortin receptor-specific agents disclosed herein, and the methods for selecting such agents, are further characterized in that the agents in general do not significantly induce a sexual response in a mammal, such as a penile erection response in a male. A number of melanocortin-receptor specific compounds and agents heretofore evaluated for use in treatment of obesity and related conditions have had unacceptable side effects relating to initiation or induction of a sexual response, including a penile erection response in males. In part, compounds of the invention, and compounds selected by methods of the invention, do not generally induce a sexual response.

The methods disclosed herein may be employed with any compound believed or suspected to bind to a melanocortin receptor, preferably to bind to MC4-R, and more preferably to bind to hMC4-R. Thus the methods and this invention may be employed with peptide, peptidomimetic, metallopeptide, small molecule, ring-core small molecule and other compounds known in the art, including compounds hereafter developed. While the invention has been exemplified with the compounds disclosed herein, the invention is not limited to such compounds, and may be applied to other compounds, which compounds may significantly differ from the compounds disclosed herein.

With respect to the compounds disclosed herein, the methods of synthesis and purification, methods of testing, formulas and definitions of classes of compounds and specific compounds disclosed in the following patent applications are relevant to the practice of the invention: U.S. patent application Ser. No. 11/557,408, entitled N-Alkylated Cyclic Peptide Melanocortin Agonists, filed on Nov. 7, 2006; U.S. patent application Ser. No. 11/464,069, entitled Melanocortin Receptor-Specific piperazine and Keto-piperazine Compounds, filed on Aug. 11, 2006; U.S. patent application Ser. No. 11/464,051, entitled Substituted Melanocortin Receptor-Specific Single Acyl piperazine Compounds, filed on Aug. 11, 2006; U.S. patent application Ser. No. 11/464,053, entitled Melanocortin Receptor-Specific piperazine Compounds with Diamine Groups, filed on Aug. 11, 2006; U.S. patent application Ser. No. 11/110,060, entitled Substituted Melanocortin Receptor-Specific piperazine Compounds, filed on Apr. 19, 2005; U.S. patent application Ser. No. 11/099,814, entitled Substituted Melanocortin Receptor-Specific piperazine Compounds, filed on Apr. 5, 2005; U.S. patent application Ser. No. 11/040,838, entitled Thieno[2,3-d]Pyrimidine-2,4-Dione Melanocortin-Specific Compounds, filed on Jan. 21, 2005; U.S. patent application Ser. No. 11/036,282, entitled Naphthlalene-Containing Melanocortin Receptor-Specific Small Molecules, filed on Jan. 14, 2005; U.S. patent application Ser. No. 10/837,519, entitled Melanocortin Receptor-Specific Compounds, filed on Apr. 30, 2004; U.S. patent application Ser. No. 10/762,079, entitled piperazine Melanocortin-Specific Compounds, filed on Jan. 21, 2004, and issued as U.S. Pat. No. 7,354,923 on Apr. 8, 2008; U.S. patent application Ser. No. 10/761,889, entitled Bicyclic Melanocortin-Specific Compounds, filed on Jan. 21, 2004, and issued as U.S. Pat. No. 7,326,707 on Feb. 5, 2008; International Patent Application PCT/US02/22196, entitled Linear and Cyclic Melanocortin Receptor-Specific Peptides, filed on Nov. 7, 2002; International Patent Application PCT/US02/25574, entitled Peptidomimetics of Biologically Active Molecules, filed on Aug. 12, 2002; and International Patent Application PCT/US01/50075, entitled Identification of Target-Specific Folding Sites in Peptides and Proteins, filed on Dec. 19, 2001. The specification and claims of each of the foregoing patent applications is incorporated herein by reference.

Definitions. Before proceeding further with the description of the invention, certain terms are defined as set forth herein.

The “amino acid” and “amino acids” used in this invention, and the terms as used in the specification and claims, include the known naturally occurring protein amino acids, which are referred to by their common three letter abbreviation. See generally Synthetic Peptides: A User's Guide, G A Grant, editor, W.H. Freeman & Co., New York, 1992, the teachings of which are incorporated herein by reference, including the text and table set forth at pages 11 through 24. Conventional amino acid residues have their conventional meaning as given in Chapter 2400 of the Manual of Patent Examining Procedure, 8^{th} Ed. Thus, “Nle” is norleucine, “Asp” is aspartic acid, “His” is histidine, “D-Phe” is D-phenylalanine, “Arg” is arginine, “Trp” is tryptophan, “Lys” is lysine, “Gly” is glycine, “Pro” is proline, “Tyr” is tyrosine, “Ser” is serine and so on.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and one or more pharmaceutically acceptable carriers, and optionally one or more pharmaceutically active ingredients and agents.

A variety of amino acids, chemicals and compounds are employed in this invention, and the following abbreviations have the meanings given:

Ac acetyl group CH₃CO—

Boc tertiary butyloxycarbonyl

Cbz benzyloxycarbonyl

DMF N,N-dimethylformamide

DMSO dimethylsulfoxide

Fmoc 9-fluorenylmethoxycarbonyl

HEPES 4-(2-hydroxyethyl)1-piperazineethanesulfonic acid

Heptanoyl CH₃—(CH₂)₅CO—

Nal 1 3-(1-naphthyl)alanine

Nal 2 3-(2-naphthyl)alanine

2-Naphthylacetyl 2-naphthyl-CH₂CO—

Phe(4-Cl) 4-chloro-phenylalanine

Phe(2,4-diCl) 2,4,-dichloro-phenylalanine

Prt A protecting group, such as Boc, Cbz or Fmoc

Ser(Bzl) O-benzyl-serine

Thr(Bzl) O-Benzyl-threonine

By a melanocortin receptor “agonist” is meant an endogenous or drug substance or compound, including a compound of this invention, that can interact with a melanocortin receptor and initiate a pharmacological response, including but not limited to an ascertainable functional activity, for example adenylyl cyclase activation, characteristic of the melanocortin receptor. By a melanocortin receptor “antagonist” is meant a drug or a compound, including a compound of this invention, that opposes the melanocortin receptor-associated responses normally induced by a melanocortin receptor agonist agent, but without itself initiating a pharmacological response characteristic of the melanocortin receptor, such as increasing or decreasing adenylyl cyclase activation. By a melanocortin receptor “inverse agonist” is meant a drug or a compound, including a compound of this invention, which is an antagonist with respect to an agonist, and which further by itself induces or initiates a pharmacological response characteristic of the melanocortin receptor, such as reducing basal or constitutive adenylyl cyclase activation. By a melanocortin receptor “protean agonist” is meant a drug or a compound, including a compound of this invention, which acts as either an inverse agonist or an agonist, depending on the constitutive activity of the MC4-R, either promoting a switch to a less active conformation or enriching the active conformation. In general, inverse and protean agonists are discussed at length in Kenakin, T. Inverse, protean, and ligand-selective agonism: matters of receptor conformation. The FASEB Journal, 15:598-611, 2001, incorporated here by reference as if set forth in full.

By “α-MSH” is meant the peptide Ac-Ser-Tyr-Ser-Met-Glu-His-Phe-Arg-Trp-Gly-Lys-Pro-Val-NH₂ (SEQ ID NO:1) and analogs and homologs thereof, including without limitation NDP-α-MSH.

By “NDP-α-MSH” is meant the peptide Ac-Ser-Tyr-Ser-Nle-Glu-His-D-Phe-Arg-Trp-Gly-Lys-Pro-Val-NH₂ and analogs and homologs thereof.

A compound that attenuates binding is a compound or drug prevents or inhibits the binding of α-MSH to melanocortin receptors, and preferably MC4-R, or decreases the binding affinity of α-MSH to melanocortin receptors, and preferably MC4-R. Compounds of this invention attenuate the binding of α-MSH to melancortin receptors. For attenuation of α-MSH binding, it is preferable if the compound being tested inhibits at least about 70% of α-MSH binding, such as iodinated NDP-α-MSH binding in a competitive inhibition assay, and more preferably inhibits at least about 85% of α-MSH, and most preferably inhibits at least about 95% or greater of α-MSH. The percent inhibition of binding can be readily determined by those skilled in the art by competition and other inhibition assays, including the methods disclosed herein and other comparable methods.

By “EC₅₀” is meant the molar concentration of an agonist, including a partial agonist, which produced 50% of the maximum possible response for that agonist. By way of example, a test compound which, at a concentration of 72 nM, produces 50% of the maximum possible response for that compound as determined in a cAMP assay in an MC4-R cell expression system has an EC₅₀ of 72 nM. Unless otherwise specified, the molar concentration associated with an EC₅₀ determination is in nanomoles per liter (nM).

By “Ki (nM)” is meant the equilibrium inhibitor dissociation constant representing the molar concentration of a competing compound that binds to half the binding sites of a receptor at equilibrium in the absence of radioligand or other competitors. In general, the numeric value of the Ki is inversely correlated to the affinity of the compound for the receptor, such that if the Ki is low, the affinity is high. Ki may be determined using the equation of Cheng and Prusoff (Cheng Y., Prusoff W. H., Biochem. Pharmacol. 22: 3099-3108, 1973):

$${K}_{i} = \frac{{EC}_{50}}{1 + \frac{[ ligand ]}{{K}_{D}}}$$

where “ligand” is the concentration of radioligand and K_{D} is an inverse measure of receptor affinity for the radioligand which produces 50% receptor occupancy by the radioligand. Unless otherwise specified, the molar concentration associated with a Ki determination is in nM. Ki may be expressed in terms of specific receptors (e.g., MC1-R, MC3-R, MC4-R or MC5-R) and specific ligands (e.g., α-MSH).

By “inhibition” is meant the percent attenuation, or decrease in receptor binding, in a competitive inhibition assay compared to a known standard. Thus, by “inhibition at 1 μM (NDP-α-MSH)” is meant the percent decrease in binding of NDP-α-MSH by addition of a determined amount of the compound to be tested, such as 1 μM of a test compound, such as under the assay conditions hereafter described. By way of example, a test compound that does not inhibit binding of NDP-α-MSH has a 0% inhibition, and a test compound that completely inhibits binding of NDP-α-MSH has a 100% inhibition. Typically, as described hereafter, a radio assay is used for competitive inhibition testing, such as with I¹²⁵-labeled NDP-α-MSH. However, other methods of testing competitive inhibition are known, including use of label or tag systems other than radioisotopes, and in general any method known in the art for testing competitive inhibition may be employed in this invention. It may thus be seen that “inhibition” is one measure to determine whether a test compound attenuates binding of α-MSH to melanocortin receptors.

By “pA₂” is meant a logarithmic measure of the potency of a compound that is an antagonist, including a partial antagonist, or the negative log of the concentration of the antagonist which produces a 2-fold shift in the concentration-response curve for an agonist. The pA₂ value is typically determined by the intercept on the y-axis of the extrapolated line in a Schild plot plotting the log (concentration ratio—1) against the log (antagonist concentration). See generally Schild, H. O. pA, a new scale for the measurement of drug antagonism. Br. J. Pharmacol. 2:189-206, 1947; and Arunlakshana, O. and Schild, H. O. Some quantitative uses of drug antagonists. Br. J. Pharmacol. 14:48-58, 1949.

By “binding affinity” is meant the ability of a compound or drug to bind to its biological target, expressed herein as Ki (nM).

By “intrinsic activity” is meant the maximal functional activity achievable by a compound in a specified melanocortin receptor expressing cell system, such as the maximal stimulation of adenylyl cyclase. The maximal stimulation achieved by α-MSH or NDP-α-MSH is designated as an intrinsic activity of 1.0 (or 100%) and a compound capable of stimulating half the maximal activity that of α-MSH or NDP-α-MSH is designated as having an intrinsic activity of 0.5 (or 50%). A compound of this invention that under assay conditions described herein has an intrinsic activity of 0.7 (70%) or higher is classified as an agonist, a compound with intrinsic activity between 0.1 (10%) and 0.7 (70%) is classified as a partial agonist, and a compound with intrinsic activity below 0.1 (10%) is classified as inactive or having no intrinsic activity. The intrinsic activity may be determined with respect to a low density melanocortin receptor expression system, a high density melanocortin receptor expression system, or a melanocortin receptor expression system which is not made, designed or selected so as to be either a low density or high density expression system.

In general, “functional activity” is a measure of the signaling of a receptor, or measure of a change in the receptor associated with signaling of the receptor, such as a melanocortin receptor, and in particular MC4-R or hMC4-R, upon activation by a compound. Melanocortin receptors initiate signal transduction through activation of heterotrimeric G proteins. In one aspect, melanocortin receptors signal through Gα_{S}, which catalyzes production of cAMP by adenylyl cyclase. Thus determination of stimulation of adenylyl cyclase, such as determination of maximal stimulation of adenylyl cyclase, is one measure of functional activity, and is the primary measure exemplified herein. However, it is to be understood that alternative measures of functional activity may be employed in the practice of this invention, and are specifically contemplated and included within the scope of this invention. Thus, in one example intracellular free calcium may be measured, such as reported by and using the methods disclosed in Mountjoy K. G. et al., Melanocortin receptor-medicated mobilization of intracellular free calcium in HEK293 cells. Physiol Genomics 5:11-19, 2001, or Kassack M. U. et al., Functional screening of G protein-coupled receptors by measuring intracellular calcium with a fluorescence microplate reader. Biomol Screening 7:233-246, 2002. It is also possible to measure activation by measurement of the production of inositol triphosphate or diacylglycerol from phosphatidylinositol 4,5-biphosphate, such as by use of radioassays. Yet another measure of functional activity is receptor internalization, resulting from activation of regulatory pathways, such as using the methods disclosed in Nickolls S. A. et al., Functional selectivity of melanocortin 4 receptor peptide and nonpeptide agonists: evidence for ligand specific conformational states. J Pharm Exper Therapeutics 313:1281-1288, 2005. Yet another measure of functional activity is the exchange, and exchange rate, of nucleotides associated with activation of a G protein receptor, such as the exchange of GDP (guanosine diphosphate) for GTP (guanosine triphosphase) on the G protein α subunit, which may be measured by any number of means, including a radioassay using guanosine 5′-(γ-[³⁵S]thio)-triphosphate, as disclosed in Manning D. R., Measures of efficacy using G proteins as endpoints: differential engagement of G proteins through single receptors. Mol Pharmacol 62:451-452, 2002. Various gene-based assays have been developed for measuring activation of G-coupled proteins, such as those disclosed in Chen W. et al., A calorimetric assay from measuring activation of Gs- and Gq-coupled signaling pathways. Anal Biochem 226:349-354, 1995; Kent T. C. et al., Development of a generic dual-reporter gene assay for screening G-protein-coupled receptors. Biomol Screening, 5:437-446, 2005; or Kotarsky K. et al., Improved receptor gene assays used to identify ligands acting on orphan seven-transmembrane receptors. Pharmacology & Toxicology 93:249-258, 2003. The calorimetric assay of Chen et al. has been adapted for use in measuring melanocortin receptor activation, as disclosed in Hruby V. J. et al., Cyclic lactam α-melanocortin analogues of Ac-Nle⁴-cyclo[Asp⁵, D-Phe⁷, Lys¹⁰] α-melanocyte-stimulating hormone-(4-10)-NH₂ with bulky aromatic amino acids at position 7 shows high antagonist potency and selectivity at specific melanocortin receptors. J Med Chem 38:3454-3461, 1995. In general, functional activity may be measured by any method, including methods of determining activation and/or signaling of a G-coupled receptor, and further including methods which may be hereafter developed or reported. Each of the foregoing articles, and the methods disclosed therein, is incorporated here by reference as if set forth in full.

Clinical Applications. The compounds disclosed herein and/or selected by the methods disclosed herein can be used for both medical applications and animal husbandry or veterinary applications. Typically, the product is used in humans, but may also be used in other mammals. The term “patient” is intended to denote a mammalian individual, and is so used throughout the specification and in the claims. The primary applications of this invention involve human patients, but this invention may be applied to laboratory, farm, zoo, wildlife, pet, sport or other animals.

In the practice of the method of this invention, compounds may be employed that are MC4-R agonists, partial agonists, antagonists, inverse agonists, or protean agonists functionally inactive in low density MC4-R expression systems, yet with demonstrated efficacy in animal models in modifying energy metabolism and feeding behavior. Thus, in one embodiment the compounds are inactive or have no intrinsic activity with respect to MC4-R at low densities, but bind MC4-R with high affinity and, in some instances, selectivity, and further have demonstrated efficacy, in animal models, in modifying energy metabolism and feeding behavior.

Formulations. The compounds may be formulated by any means known in the art, including but not limited to tablets, capsules, caplets, suspensions, powders, lyophilized forms and aerosols/aerosolizable forms and may be mixed and formulated with buffers, binders, stabilizers, anti-oxidants and other agents known in the art. The compounds may be administered by any systemic or partially systemic means such as those known in the art, including but not limited to intravenous injection, subcutaneous injection, administration through mucous membranes, oral administration, dermal administration, skin patches, aerosols and the like.

The invention further provides a pharmaceutical composition and method for the use thereof that includes a compound of this invention and a pharmaceutically acceptable carrier. The compounds may thus be formulated or compounded into pharmaceutical compositions that include at least one compound of this invention, or a compound selected by a method of this invention, together with one or more pharmaceutically acceptable carriers, including excipients, such as diluents, carriers and the like, and additives, such as stabilizing agents, preservatives, solubilizing agents, buffers and the like, as may be desired. Formulation excipients may include polyvinylpyrrolidone, gelatin, hydroxy cellulose, acacia, polyethylene glycol, mannitol, sodium chloride or sodium citrate. For injection or other liquid administration formulations, water containing at least one or more buffering constituents is suitable, and stabilizing agents, preservatives and solubilizing agents may also be employed. For solid administration formulations, any of a variety of thickening, filler, bulking and carrier additives may be employed, such as starches, sugars, fatty acids and the like. For pharmaceutical formulations, it is also contemplated that any of a variety of measured-release, slow-release or time-release formulations and additives may be employed, such that the dosage may be formulated so as to effect delivery of a compound of this invention over a period of time.

The compounds of this invention, including compounds not disclosed herein but selected by a method of this invention, may be in the form of any pharmaceutically acceptable salt. Acid addition salts of the compounds of this invention are prepared in a suitable solvent from the compound and an excess of an acid, such as hydrochloric, hydrobromic, sulfuric, phosphoric, acetic, trifluoroacetic, maleic, succinic or methanesulfonic acids. The acetate salt form is especially useful. Where the compounds of this invention include an acidic moiety, suitable pharmaceutically acceptable salts may include alkali metal salts, such as sodium or potassium salts, or alkaline earth metal salts, such as calcium or magnesium salts.

The compounds and pharmaceutical compositions may be administered by injection, which injection may be intravenous, subcutaneous, intramuscular, intraperitoneal or by any other means known in the art. In general, any route of administration by which the compounds of this invention are introduced across an epidermal layer of cells may be employed. Administration means may include administration through mucous membranes, buccal administration, oral administration, dermal administration, inhalation administration, nasal administration and the like. The dosage for treatment is administration, by any of the foregoing means or any other means such as those known in the art, of an amount sufficient to bring about the desired therapeutic effect.

Therapeutically Effective Amount. In general, the actual quantity of compound administered to a patient will vary between fairly wide ranges depending upon the mode of administration, the formulation used, and the response desired. This may readily be determined by one of ordinary skill in the art through means such as pharmacokinetic studies, plasma half-life studies, dose escalation studies, and the like. The dosage for treatment is administration, by any of the foregoing means or any other means known in the art, of an amount sufficient to bring about the desired therapeutic or biological effect. Thus, a therapeutically effective amount includes an amount of a compound or pharmaceutical composition of this invention that is sufficient to induce the desired therapeutic or biological effect.

The compounds of this invention are highly active in modifying energy metabolism and feeding behavior. For example, the compound can be administered at 0.01, 0.05, 0.1, 0.5, 1, 5, 10, 50, 100, or 500 μg/kg body weight, depending on the specific compound selected, the desired therapeutic response, the route of administration, the formulation and other factors known to those of skill in the art. Conventional dose response studies and other pharmacological means may be employed to determine the optimal dose for a desired effect with a given compound, given formulation and given route of administration.

Combination Therapy and Weight Regulation. It is also possible and contemplated to use compounds of this invention, or compounds selected by a method of this invention, in combination with other drugs or agents for treatment of various weight and feeding-related disorders. Any of the combinations disclosed in U.S. patent application Ser. No. ______ (Attorney Docket No. 056291-5365), entitled Compounds and Methods for Treating Obesity, which is filed concurrently herewith and which claims priority to U.S. Provisional Patent Application Ser. No. 60/712,282, may be employed here, and the teachings thereof are specifically incorporated herein by reference.

### Assays and Animal Models.

Selected compounds were tested in assays to determine binding and functional status, and were tested in animal models of penile erection and feeding behavior, as discussed below. The following assays and animal models were employed, with modifications as discussed in the examples.

Competitive inhibition assay using [I¹²⁵]-NDP-α-MSH. A competitive inhibition binding assay was conducted using membranes prepared from hMC3-R, hMC4-R, hMC5-R, and B-16 mouse melanoma cells (containing MC1-R) in 25 mM HEPES buffer (pH 7.5) with 100 mM NaCl, 2 mM CaCl₂, 2 mM MgCl₂, 0.3 mM 1,10-phenanthroline, and 0.2% bovine serum albumin, and the radiolabeled ligand [I¹²⁵]-NDP-α-MSH (New England Nuclear). The ligand concentration used was 0.4 nM for MC3-R and MC5-R, 0.2 nM for MC4-R and 0.1 nM for B16-F10 (MC1-R) membranes. In certain experiments, hMC1-R was also employed. The assay tube also contained a chosen concentration of the test compound of this invention, typically a 1 μM concentration, for determining its efficacy in inhibiting the binding of [I¹²⁵]-NDP-α-MSH to its receptor. Non-specific binding was measured by inhibition of binding of [I¹²⁵]-NDP-α-MSH in the assay with the presence of 1 μM α-MSH.

The assay mixture was incubated for 90 minutes at room temperature, then filtered and the membranes washed three times with ice cold buffer. The filter was dried and counted in a gamma counter for remaining radioactivity bound to the membranes. 100% specific binding was defined as the difference in radioactivity (cpm) bound to cell membranes in the absence and presence of 1 μM α-MSH. The cpm obtained in presence of test compounds were normalized with respect to 100% specific binding to determine the percent inhibition of [I¹²⁵]-NDP-α-MSH binding. Each assay was conducted in triplicate and the actual mean values are described, with results less than 0% reported as 0%.

High and Low Receptor Density Systems. A tetracycline-regulated mammalian expression system that uses regulatory elements from the E. coli Tn 10-encoded tetracycline (Tet) resistance operon was employed (T-REx™ System, Invitrogen). The T-REx™ System Manual, published by Invitrogen, is incorporated by reference. By use of the T-REx™ System, expression of the gene of interest, the human MC4-R gene, was repressed in the absence of tetracycline or doxycycline and induced in the presence of tetracycline or doxycycline, with receptor density being dependent on the concentration of tetracycline or doxycycline. See generally Yao F. et al: Tetracycline Repressor, tetR, Rather than the tetR-Mammalian Cell Transcription Factor Fusion Derivatives, Regulates Inducible Gene Expression in Mammalian Cells. Hum. Gene Ther. 9:1939-1950 (1998), incorporated here by reference. Regulation was based on the binding of tetracycline or doxycycline, a derivative of tetracycline, to the Tet repressor and derepression of the promoter controlling expression of the human MC4-R gene. In general tetracycline or related regulators, such as tetracycline-derivatives, may be used to induce the gene expression. An inducible expression plasmid for expression of human MC4-R gene under the control of the strong human cytomegalovirus immediate-early (CMV) promoter and two tetracycline operator 2 (TetO₂) sites was employed, together with a regulatory plasmid, pcDNA6/TR, which encodes the Tet repressor (TetR) under the control of the human SV40 promoter. Thus, expression of the human MC4-R gene from the inducible expression vector was controlled by the strong CMV promoter into which 2 copies of the tet operator 2 (TetO₂) sequence had been inserted in tandem. The TetO₂ sequences consisted of 2 copies of the 19 nucleotide sequence, 5′-TCCCTATCAGTGATAGAGA-3′ (SEQ ID NO: 2) separated by a 2 base pair spacer. Each 19 nucleotide TetO₂ sequence served as the binding site for 2 molecules of the Tet repressor. The expression vector pcDNA4/TO/MC4R was introduced into TREx-293 (Invitrogen, R710-07), a cell line stably expressing the tetracycline repressor pcDNA6/TR using lipofectamin 2000 reagent (Invitrogen, 11668-019). Alternatively, both the expression vector and repressor are introduced into host cells by standard transformation or transfection methods.

The receptor density at different doxycycline concentrations was quantified in terms of binding per mg of protein derived from cell membranes using [I¹²⁵]-NDP-α-MSH in receptor binding saturation studies to determine a Bₘₐₓ value. Table 1 below quantifies the receptor density:

TABLE 1

| Doxycycline | Bₘₐₓ ± SD | K_{D(app)} ± SD | |
|---|---|---|---|
| (ng/mL) | (fmol/mg) | (pM) | n |
| 0.1 | (62 ± 13) | (114 ± 57) | 8 |
| 1 | 207 ± 128 | 118 ± 37 | 11 |
| 10 | 10113 ± 2538 | 139 ± 41 | 11 |

By comparison, transformed or transfected cells not under control of a tetracycline- or doxycycline-regulated expression system, such as cells used under the heading “Competitive inhibition assay using [I¹²⁵]-NDP-α-MSH” above and elsewhere in this application, typically resulted in a Bₘₐₓ value of approximately 400 fmol/mg using [I¹²⁵]-NDP-α-MSH.

In other studies, up to 100 ng/mL of doxycycline was employed. However, analysis of data showed that the difference between 0.1 and 10 ng/mL of doxycycline was sufficient to differentiate binding as hereafter described.

In general, any cell line which may be reproducibly transformed or transfected, stably or transiently, may be employed. Thus, conventional mammalian cell lines, such as HEK-293 and CHO (Chinese hamster ovary) cell lines may readily be employed. Similarly, any controlled expression system may be employed, and lines that express high and low densities of receptors selected. Conventionally such expression systems are under the control of a drug, such as an antibiotic such as tetracycline. However, any expression system, such as those known in the art, which is able to reproducibly vary the expression level of receptors may be employed in the practice of the invention, and it is intended to encompass all such methods, cells, and like within the scope of the invention. It will be apparent to those skilled in the art that transformation, transient transfection or stable transfection or a combination thereof may be employed to express the necessary cell-expressed components of the assays of the invention, including the melanocortin receptor, within the cells which are preferably but not necessarily mammalian cells. Thus, for example, the cells of the assay may comprise a transgene encoding and permitting expression, variable or constitutive as needed, of the melanocortin receptor. The transgene may be stably integrated in the genome of the cells or may be present on a transient expression vector. In one alternative, an endogenous melanocortin receptor gene is placed under control of inducible gene regulatory elements by stably introducing the gene elements into a cell such that they are operably linked to the endogenous melanocortin receptor gene.

In an alternative embodiment, any selection method may be employed in a population of cells that are transformed or transfected to express a melanocortin receptor, with cells expressing low densities and high densities selected. Selection, particularly over successive generations, may result in cell lines which express the desired densities, and are sufficiently stable to permit reproducible experimentation.

As used in the specification and claims, a “low density melanocortin receptor system” includes, but is not limited to, a transfected or transformed cell gene expression system, preferably under the control of a gene expression variable regulatory system such as described above, which has a melanocortin receptor density measured in receptor binding saturation experiments that is at least ten times lower, and preferably is more than ten times lower, such as about 50 to 100 times lower, than the melanocortin receptor density in a “high density melanocortin receptor system.” A “high density melanocortin receptor system” includes, but is not limited to, a transfected or transformed cell gene expression system, preferably under the control of a gene expression variable regulatory system such as described above.

A “low density MC4-R system” is a low density melanocortin receptor system wherein the melanocortin receptor is MC4-R, preferably human MC4-R (hMC4-R). A “high density MC4-R system” is a high density melanocortin receptor system wherein the melanocortin receptor is MC4-R, preferably hMC4-R.

A transformed cell system not employing a variable regulatory system, such as transformation of HEK-293 cells with a human MC4-R gene, may also be employed in the practice of the invention, and as such is neither a low density melanocortin receptor system nor a high density melanocortin receptor system. Any suitable transformation or transfection methodology, including without limitation those for stable transfection and those for transient transfection, or reagents may be used, such as use of Polyfect (QIAGEN, Valencia, Calif.) generally following the manufacturer's instructions or as described by Gantz I., Miwa H., et al. Molecular cloning, expression, and gene localization of a fourth melanocortin receptor. J. Biol. Chem. 268:15174-15179. 1993. Such a transformed system is called herein a “normal melanocortin receptor density system,” and is “normal” in that the system is transformed at the usual rate for the transformed methodology and reagents employed, and is not under the control of a gene expression variable regulatory system.

In a preferred embodiment of the present invention, a low density melanocortin receptor system has a receptor density, such as density of hMC4-R, which is lower than receptor density typically present in a normal melanocortin receptor density system. Preferably the receptor density is between about 5 and 20 times lower, more preferably between about 5 and 10 times lower. It may thus be seen that if a normal melanocortin receptor density system, such as HEK-293 cells transfected or transformed with a human MC4-R gene, has a receptor density expressed as a Bₘₐₓ value of approximately 400 fmol/mg using [I¹²⁵]-NDP-α-MSH in receptor binding saturation experiments, that a low density melanocortin receptor system transformed with a human MC4-R gene would have a receptor density expressed as a Bₘₐₓ value of between approximately 80 and 20 fmol/mg using [I¹²⁵]-NDP-α-MSH, preferably between approximately 80 and 40 fmol/mg using [112]-NDP-α-MSH. Thus, a system having cells with a Bₘₐₓ±SEM (fmol/mg) of about 62±13, as used herein, is a low density melanocortin receptor system using hMC4-R.

In a related preferred embodiment of the present invention, a high density melanocortin receptor system has a receptor density, such as density of MC4-R, which is higher than receptor density in a normal melanocortin receptor density system. Preferably the receptor density is between about 10 and 30 times higher, more preferably between about 10 and 25 times higher. However, an even higher receptor density may be employed. It may thus be seen that if a normal melanocortin receptor density system, such as HEK-293 cells transfected or transformed with a human MC4-R gene, has a receptor density expressed as a Bₘₐₓ value of approximately 400 fmol/mg using [I¹²⁵]-NDP-α-MSH, that a high density melanocortin receptor system transformed with a human MC4-R gene would have a receptor density expressed as a Bₘₐₓ value of between approximately 4,000 and 12,000 fmol/mg using [I¹²⁵]-NDP-α-MSH in receptor binding saturation experiments, preferably between approximately 4,000 and 10,000 fmol/mg using [I¹²⁵]-NDP-α-MSH. Thus, a system having cells with a Bₘₐₓ±SEM (fmol/mg) of about 10,113±2538, as used herein, is a high density melanocortin receptor system using hMC4-R.

It may further be seen that the low density melanocortin receptor system using hMC4-R expressing cells with a Bₘₐₓ±SEM (fmol/mg) of about 62±13 has a receptor density that is about 163 times (ranging from about 101 to 258 times) lower than the receptor density of the high density melanocortin receptor system using hMC4-R expressing cells with a Bₘₐₓ±SEM (fmol/mg) of about 10,113±2538.

It is to be understood that in biological cell gene expression systems as described herein there is inherently some drift in both the density of receptors and responsiveness of the systems. As is known, even normal melanocortin receptor density system cells may have to be re-established on a regular basis to maintain a reasonably constant response. Additionally, where relative efficacy is to a biologically active peptide, it is possible and known that relative efficacy compared to such peptide, such as relative efficacy compared to NDP-α-MSH, may fluctuate from one preparation to another on different days, but generally without change in the rank order for a set of test compounds.

General method for EC₅₀ determination in functional activity assay. Functional evaluation of compounds at melanocortin receptors was performed by measuring the accumulation of intracellular cAMP in HEK-293 cells expressing human MC3-R, MC4-R or MC5-R, and in B-16 mouse melanoma cells expressing MC1-R. Cells suspended in Earle's Balanced Salt Solution containing 10 mM HEPES (pH 7.5), 5 mM MgCl₂, 1 mM glutamine, 0.1% albumin and 0.6 mM 3-isobutyl-1-methyl-xanthine, a phosphodiesterase inhibitor, were plated in 96 well plates at a density of 0.5×10⁵ cells per well. Cells were incubated with the test compounds in the presence or absence of α-MSH for 1 hour at 37° C. cAMP levels in the cell lysates were measured using an EIA kit (Amersham). Data analysis and EC₅₀ values were determined using nonlinear regression analysis with Prism Graph-Pad software.

Functional status. The agonist/antagonist status with respect to MC1-R, MC-3R, MC4-R and MC5-R of certain compounds of the invention was determined. Antagonistic activity was determined by measuring the inhibition of α-MSH-induced or NDP-α-MSH-induced cAMP levels following exposure to graded doses of compounds as in the preceding descriptions.

Assay for agonist. Evaluation of the molecules to elicit a functional response in HEK-293 cells expressing MC4-R was accomplished by measuring the accumulation of intracellular cAMP following treatment. Confluent HEK-293 cells expressing MC4-R receptors were detached by enzyme free cell suspension buffer. Cells were suspended in Earle's Balanced Salt Solution containing 10 mM HEPES (pH 7.5), 1 mM MgCl₂, 1 mM glutamine, 0.5% albumin and 0.3 mM 3-isobutyl-1-methyl-xanthine (IBMX), a phosphodiesterase inhibitor. The cells were plated in a 96 well plates at a density of 0.5×10⁵ cells per well and pre-incubated for 30 minutes. Test compounds (dissolved in dimethylsulfoxide (DMSO) at a concentration range of 0.05-5000 nM) were added in a total assay volume of 200 μL and cells were incubated for 1 hour at 37° C. The concentration of DMSO was always held at 1% in the assay mixture. NDP-α-MSH was used as the reference agonist. At the end of the incubation period the cells were disrupted by the addition of 50 μL of lysis buffer from a cAMP EIA kit (Amersham). Complete rupture of the cells was obtained by pipetting the cells multiple times. cAMP levels in the cell lysates were measured after appropriate dilution using the EIA kit (Amersham) method. Data analysis and EC₅₀ values were determined by using nonlinear regression analysis with Prism Graph-Pad software. Compounds at a concentration of 5000 nM that had a response ratio compared to NDP-α-MSH of 0.7 (70%) and above were classified as full agonists. Compounds with a ratio from 0.1 to 0.7 (10% to 70%) were classified as partial agonists. Compounds with a response ratio of less than 0.1 (10%) were evaluated for antagonistic activity.

Assay for Neutral Antagonist. Compounds with a high affinity for binding to MC4-R membranes but with less efficacy (EC₅₀>1000 nM) and low response ratio (<0.1) were analyzed for their ability to antagonize the stimulatory effect of the agonist NDP-α-MSH. These studies were carried out in HEK-293 cells expressing the MC4-R receptor. Cells were incubated with the compounds in the presence of the agonist NDP-α-MSH and the extent of antagonism was measured by the decrease in the level of intracellular cAMP. Screening the compounds for antagonists was done at a single concentration of NDP-α-MSH (1.0 nM) over a compound concentration range of 0.5-5000 nM. Studies were extended further for compounds exhibiting strong antagonism to derive the pA₂ value from Schild analysis.

Experimental details were similar to the analysis for agonistic activity described above. Briefly, cells were pre-incubated for 30 minutes with the test compounds at concentrations between 0.5 nM and 5000 nM. The cells were then stimulated with NDP-α-MSH at a concentration of 1 nM for 1 hour. For Schild analysis, the interactions were studied using at least 3 concentrations of the compounds, separated by a log unit, over a full range of the agonist (0.005-5000 nM). cAMP levels was measured in the cell lysates after appropriate dilution. Nonlinear regression analysis with Prism Graph-Pad software was used for Schild analysis and to obtain EC₅₀ values. pA₂ values were derived from the Schild plot of the data.

Cell culture and doxycycline induction. HEK-293 cells that express hMC4-R using a doxycycline-regulated expression system were grown in DMEM (Gibco) supplemented with 10% fetal bovine serum, 250 μg/mL Zeocin (Invitrogen), 5 μg/mL Blasticidin (Invitrogen) and 2 mM glutamine (Invitrogen.). Cells were maintained at 37° C. in an environment of 5% CO₂. To induce receptor expression, cells (at approximately 85% confluency) were exposed to doxycycline for 18 hours.

Membrane preparation and radioligand binding. After doxycycline induction, media containing doxycycline was removed and cells were washed with cold PBS. To disrupt cells from culture flasks, 10 mL of cold buffer (10 mM Tris, pH 7.4, 5 mM EDTA) was added to culture flasks and allowed to incubate for 10 minutes at 4° C. Dislodged cells were transferred to assay tubes and homogenized for 15 seconds with a Polytron homogenizer. The cell homogenates were centrifuged for 30 minutes at 20000 rpm. The supernatant was removed and pellets were resuspended in storage buffer (50 mM Tris, pH 7.4, 1 mM EDTA). Aliquots of 1 mL were made from each resuspended pellet and frozen at −80° C. A Bradford protein assay was used to determine protein concentration with BSA as the standard. On the day of an assay, membrane homogenates were thawed and incubated (3 μg protein/well) at 37° C. for 90 minutes with [I¹²⁵]-NDP-α-MSH at concentrations from 0.02 nM to 1 nM in 200 μL assay buffer (25 mM HEPES, pH 7.4, 100 mM NaCl, 2 mM CaCl₂, 2 mM MgCl₂, 0.3 mM 1,10 phenanthroline/DMSO and 0.2% BSA). The reaction was stopped by filtration over GF/C filters (presoaked in 0.5% polyethylenimine) with five washes of cold PBS using a Brandel harvester. Non-specific binding was determined with the addition of 1 μM NDP-αMSH. Membrane filters were punched and counted on a PerkinElmer Cobra gamma counter. Bₘₐₓ and K_{D} values were determined by Scatchard analysis.

Agonist stimulation in cells using a doxycycline-regulated expression system. After induction of MC4-R expression with doxycycline, media containing doxycycline was removed and cells were detached by incubation in enzyme-free cell suspension buffer. Assay buffer consisting of Hank's Balanced Salt Solution, 10 mM HEPES (pH 7.4), 5 mM MgCl₂, 1 mM glutamine, 1 mg/mL BSA and 0.5 mM 3-isobutyl-1-methyl-xanthine (IBMX) was added, cells were transferred to assay tubes and centrifuged for 4 minutes at 1600 rpm. Following centrifugation, cells were re-suspended in the assay buffer, transferred to 96 well plates at a density of 5 to 10×10⁴ cells per well and incubated for 10 minutes at 37° C. and 5% CO₂. Cells were exposed to test compounds (10 μM to 10 μM) for 15 minutes at 37° C. in an assay volume of 200 μL. At the end of the incubation period, cells were lysed by the addition of 50 μL of lysis buffer from a cAMP EIA kit (Amersham) or frozen at −80° C. cAMP levels in the cell lysates were measured using the Adenylyl Cyclase Activation FlashPlate Assay (Perkin Elmer). FlashPlates were incubated at room temperature for 12 hours and activity determined by a Tri-Lux Microbeta counter (Perkin Elmer). EC₅₀ and Eₘₐₓ values for the dose-response curves were determined using PRISM (GraphPad Software Inc., San Diego, Calif.).

Penile erection induction. The ability of compounds to induce penile erection (PE) in male rats was evaluated with selected compounds. Male Sprague-Dawley rats weighing 200-250 g were kept on a 12 hour on/off light cycle with food and water ad libitum. All behavioral studies were performed between 10 A.M. and 5 P.M. Groups of 4-8 rats were treated with compounds at a variety of doses via intravenous (IV) or intracerebroventricular (ICV) routes. Immediately after treatment, rats were placed into individual polystyrene cages (27 cm long, 16 cm wide, and 25 cm high) for behavioral observation. Rats were observed for 30 to 60 minutes following IV administration or 120 minutes following ICV administration, and the number of yawns, grooming bouts and PEs were recorded in 10-minute bins. Controls utilized carrier without the test compound. Mean PEs in control groups were 0.17 to 0.5 PEs/rat by IV administration in various experiments and between 1-2 PEs/rat by ICV administration in various experiments, and thus only PEs with statistically relevant increases over the mean PEs in control groups are reported as inducing PEs. A PE response in IV animals greater than the mean PEs in control groups but less than 1.0 PEs/rat, particularly with less than all animals responding, was treated as equivocal, and not necessarily distinguishable from vehicle control. In selected instances, compounds with equivocal results in the IV model were tested for penile response in an ICV model, and compounds without a statistically relevant increase over the mean PEs in ICV control groups were determined to not induce PEs.

ICV food intake and body weight change. Change in food intake and body weight was evaluated for selected compounds. Rats with indwelling intracerebroventricular cannulas (ICV rats) were obtained from Hilltop Lab Animals, Inc. (Scottdale, Pa.). Animals were individually housed in conventional plexiglass hanging cages and maintained on a controlled 12 hour on/off light cycle. Water and powdered (LabDiet, 5P00 Prolab RMH 3000) or pelleted (Harlan Teklad 2018 18% Protein Rodent Diet) food was provided ad libitum. For 1 week before treatment, 24-hour food intake and body weight change was recorded to assess a baseline for the group during vehicle treatment. The rats were dosed ICV with vehicle or selected compounds (1-3 nmol). The changes in body weight and food intake for the 24 hour period after dosing were determined. The changes in body weight and food intake for the 48 hour and 72 hour periods after dosing were also measured to determine reversal of changes in body weight and food intake effects back to baseline levels.

IV and IP food intake and body weight change. Change in food intake and body weight was evaluated for selected compounds. Male Sprague-Dawley rats or male C57BL/6 mice were utilized. Animals were individually housed in conventional plexiglass hanging cages and maintained on a controlled 12 hour on/off light cycle. Water and powdered (LabDiet, 5P00 Prolab RMH 3000) or pelleted (Harlan Teklad 2018 18% Protein Rodent Diet) food was provided ad libitum. For 1 week before treatment, 24-hour food intake and body weight change was recorded to assess a baseline for the group during vehicle treatment. The rats were dosed IV with vehicle or selected compounds (0.5-3 mg/kg, and in some cases up to 10 mg/Kg) or dosed intraperitoneally (IP) with vehicle or selected compounds (0.5-10 mg/kg, and in some cases up to 50 mg/kg). Mice were dosed IP. The changes in body weight and food intake for the 24 hour period after dosing were determined. The changes in body weight and food intake for the 48 hour and 72 hour periods after dosing were also measured to determined reversal of changes in body weight and food intake effects back to baseline levels.

Determination of mass and nuclear magnetic resonance analysis. The mass values were determined using a Waters MicroMass ZQ device utilizing a positive mode. Mass determinations were compared with calculated values and expressed in the form of mass weight plus one (M+1 or M+H).

Proton NMR data was obtained using a Bruker 300 MHz spectrometer. The spectra were obtained after dissolving compounds in a deuteriated solvent such as chloroform, DMSO, or methanol as appropriate.

Summary of cAMP Response in High and Low Receptor Density Studies. Table 2 lists the compounds employed in low density and high density receptor studies. Table 3 summarizes the data obtained with those compounds.

TABLE 2

| Compound | |
|---|---|
| or | |
| Example | |
| No. | Molecular Structure |
| NDP-α- | Ac-Ser-Tyr-Ser-Met-Glu-His-D-Phe-Arg-Trp-Gly-Lys-Pro-Val-NH₂ |
| MSH | |
| α-MSH | Ac-Ser-Tyr-Ser-Nle-Glu-His-Phe-Arg-Trp-Gly-Lys-Pro-Val-NH₂ (SEQ ID NO: 1) |
| MT-II | Ac-Nle-cyclo(-Asp-His-D-Phe-Arg-Trp-Lys)-NH₂ |
| PT-141 | cyclo 2,7[Ac-Nle-Asp-His-D-Phe-Arg-Trp-Lys]-OH |
| SHU-9119 | cyclo 2,7[Ac-Nle-Asp-His-D-Nal 2-Arg-Trp-Lys]-NH₂ |
| AgRP (83- | (AgRP (83-132)) |
| 132) | |
| 1 | NH₂—(CH₂)₆C(═O)-Ser(Bzl)-D-Phe(4-Cl)-Arg-Trp-NH₂ |
| 2 | 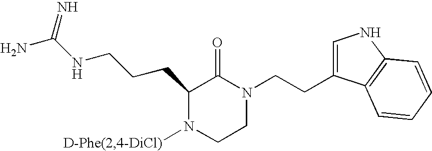 |
| 3 | 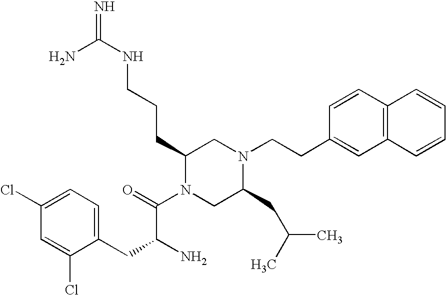 |
| 4 | 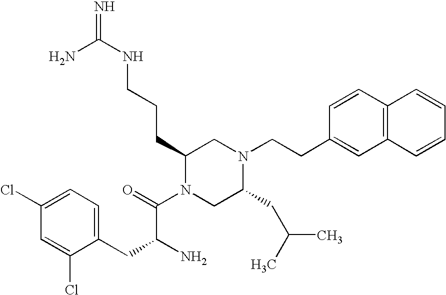 |
| 5 | 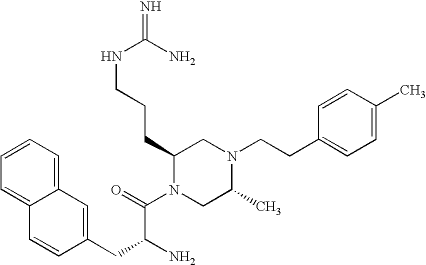 |
| 6 | 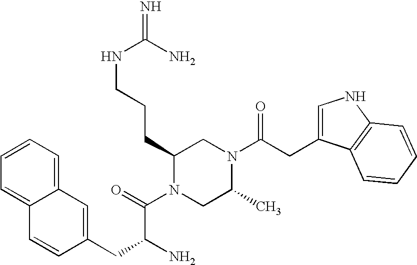 |
| 7 | 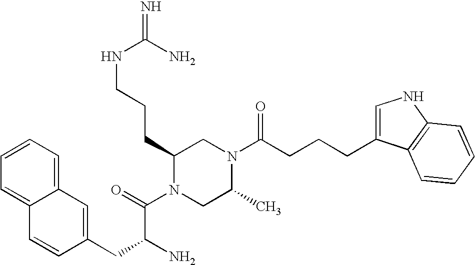 |
| 8 | 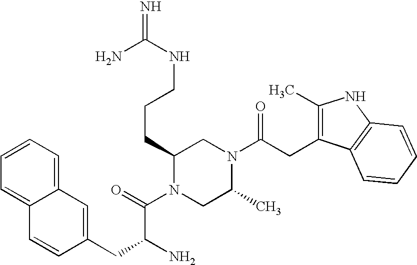 |
| 9 | 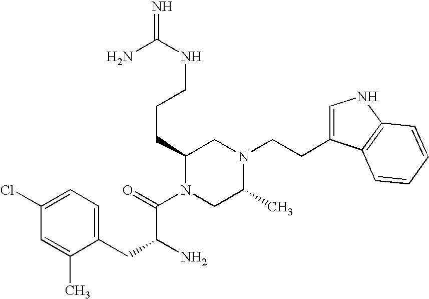 |
| 10 | 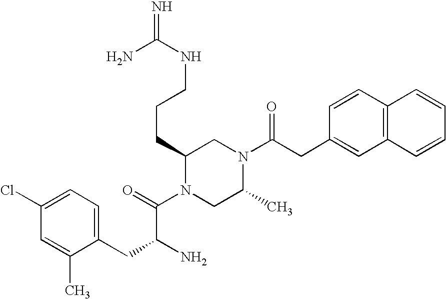 |
| 11 | 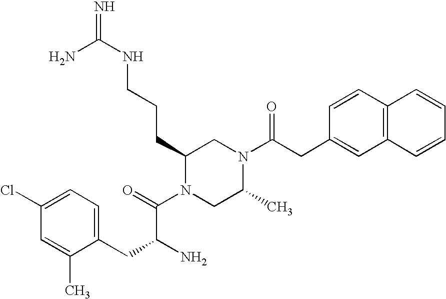 |
| 12 | 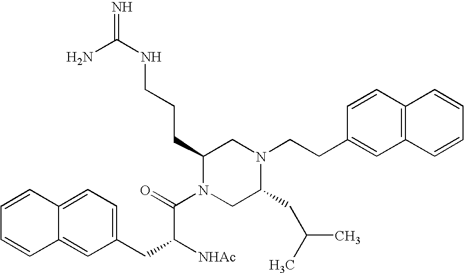 |
| 13 | NH₂—(CH₂)₆—C(═O)-Ser(Bzl)-D-Phe(4-Cl)-Arg-Trp-Asp-NH₂ |
| 14 | NH₂—(CH₂)₆—C(═O)-Ser(Bzl)-D-Phe(4-Cl)-Arg-Trp-Asp-Phe-NH₂ |
| 15 | NH₂—(CH₂)₆—C(═O)-Ser(Bzl)-D-Phe(4-Cl)-Arg-Trp-Ala-NH₂ |
| 16 | 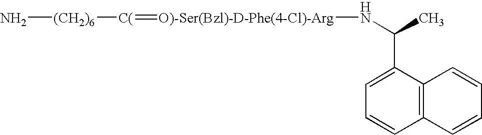 |
| 17 | 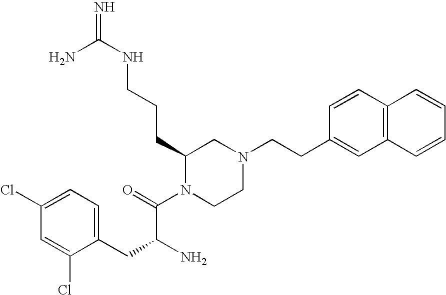 |
| 18 | 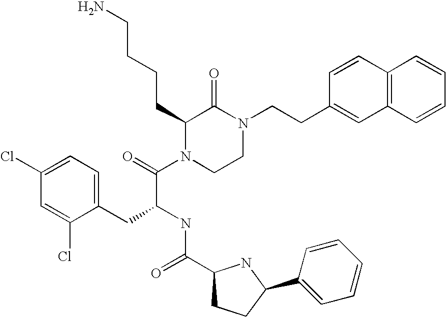 |
| 19 | 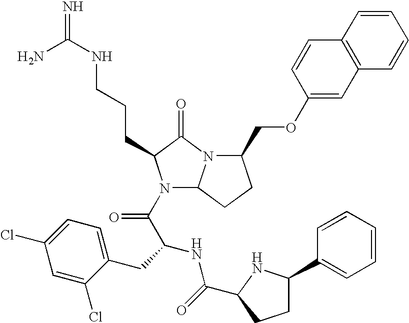 |
| 20 | 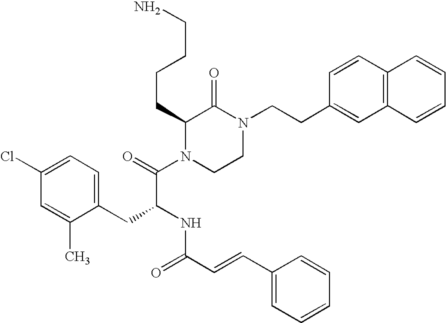 |
| 21 | 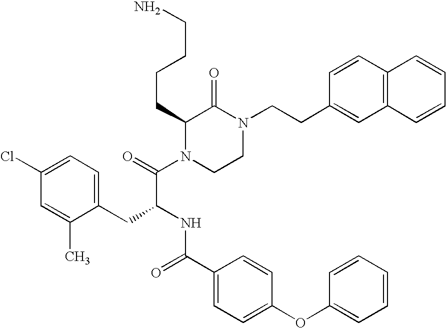 |
| 22 | 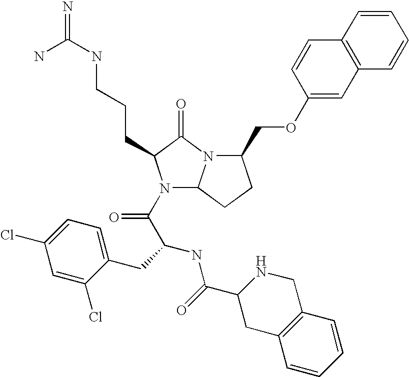 |
| 23 | heptanoyl-Thr(Bzl)-D-Phe(4-Cl)-Arg-Trp-NH₂ |
| 24 | 2-naphthylacetyl-1-amino-1-cyclohexane-carbonyl-D-Phe(4-Cl)-Arg-Trp-NH₂ |
| 25 | 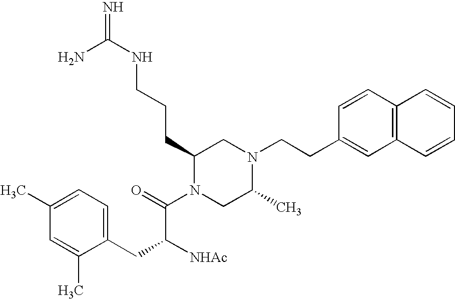 |
| 26 | 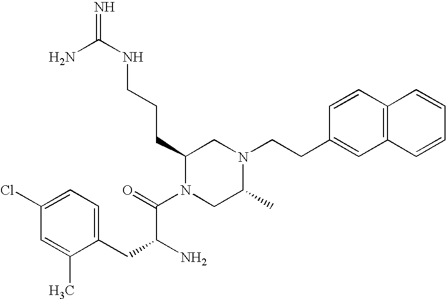 |
| 27 | 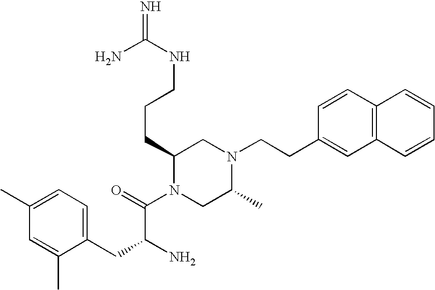 |
| 28 | 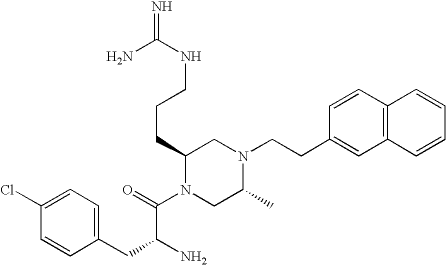 |
| 29 | 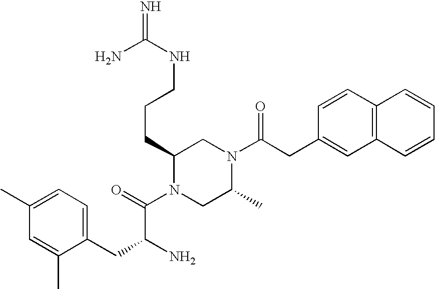 |
| 30 | 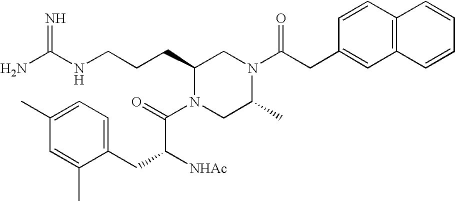 |
| 31 | 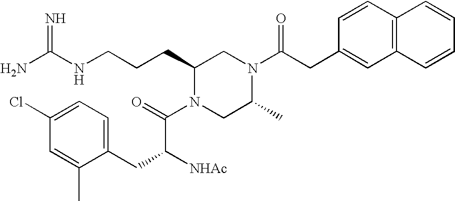 |
| 32 | 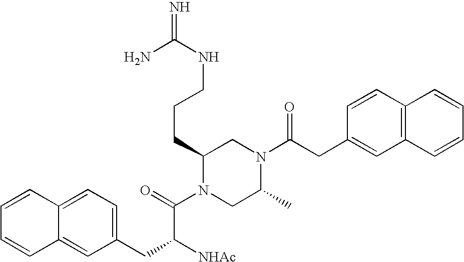 |
| 33 | 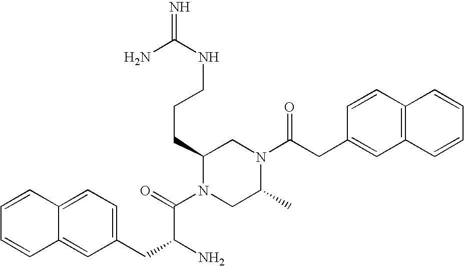 |
| 34 | 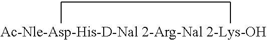 |
| 35 | 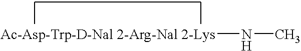 |
| 36 | 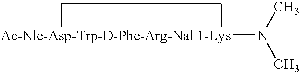 |
| 37 | 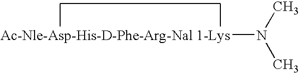 |
| 38 | 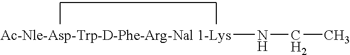 |
| 39 | 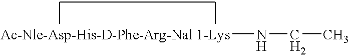 |
| 40 | 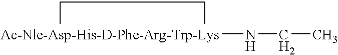 |

TABLE 3

| | 0.1 ng/mL | | 10 ng/mL | | | | | | |
|---|---|---|---|---|---|---|---|---|---|
| | Doxycycline | | Doxycycline | | | | | | |
| | Efficacy/ | | Efficacy/ | | Penile | | Food Intake | | |
| Compound | Intrinsic | EC₅₀ | Intrinsic | EC₅₀ | Erection | | ICV | Rat | Mouse |
| No. | Activity | (nM) | Activity | (nM) | ICV | IV | Rat | IV/IP | IP |
| NDP-α- | 1 | 1 | 1 | 0.5 | | | ↓ | | |
| MSH¹ | | | | | | | | | |
| α-MSH | | | 0.9 | 1 | | | ↓ | | |
| MT-II² | 0.77 | 0.5 | >1 | 0.4 | Yes** | Yes** | ↓ | | |
| PT-141 | 0.90 | 3 | 1 | 0.9 | Yes** | Yes** | ↓ | | |
| SHU-9119³ | None | | None | | No | No | ↑ | | |
| AGRP (83-132)³ | None | | None | | No | No | | ↑ | |
| 1 | None | | 0.97 | 21 | No | | ↓ | ↓ | |
| 2 | None | | 0.56 | 57 | | No | | | |
| 3 | None | | 0.26 | 61 | | | | | |
| 4 | None | | None | | | | | ↓ | |
| 5 | None | | 0.46 | 117 | | No | | | |
| 6 | None | | 0.43 | 39 | | No | | | |
| 7 | None | | 0.38 | 72 | No | | | | |
| 8 | None | | None | | No | | | | |
| 9 | None | | 0.73 | 22 | No | No | ↓ | | |
| 10 | 0.23 | 38 | 0.69 | 26 | | Yes* | | ↓ | |
| 11 | None | | 0.49 | 61 | | | | ↓ | |
| 12 | None | | None | | | | | ↓ | |
| 13 | None | | 0.72 | 34 | | No | ↓ | ↓ | |
| 14 | None | | 0.64 | 141 | | No | ↓ | ↓ | |
| 15 | None | | 0.67 | 55 | | No | ↓ | ↓ | |
| 16 | None | | 0.63 | 180 | | No | ↓ | | |
| 17 | None | | 0.67 | 847 | No | No | ↓ | ↓ | |
| 18 | None | | 0.29 | 281 | | | ↓ | | |
| 19 | None | | 0.22 | 173 | | No | ↓ | ↓ | |
| 20 | None | | 0.38 | 215 | | No | ↓ | | |
| 21 | None | | None | | | No | ↓ | | |
| 22 | None | | 0.30 | 64 | | No | ↓ | | |
| 23 | None | | 0.75 | 184 | | No | ↓ | | |
| 24 | None | | None | | | | ↓ | | |
| 25 | None | | None | | | | | ↓ | |
| 26 | None | | 0.78 | 31 | | No | ↓ | | ↓ |
| 27 | None | | 0.61 | 129 | No | No | ↓ | | ↓ |
| 28 | 0.29 | 106 | 0.75 | 24 | | No | ↓ | | ↓ |
| 29 | 0.25 | 45 | 0.77 | 34 | | Yes* | | | ↓ |
| 30 | None | | 0.18 | 51 | | No | | | ↓ |
| 31 | None | | 0.27 | 39 | | No | | | ↓ |
| 32 | None | | None | | No | No | | | ↓ |
| 33 | None | | 0.73 | 50 | | No | | | ↓ |
| 34 | None | | None | | No | | ↑ | | |
| 35 | None | | None | | No | | ↑ | | |
| 36 | None | | 0.98 | 4 | | | | | |
| 37 | 0.69 | 2.1 | 0.88 | 0.6 | | | | | |
| 38 | None | | 1.0 | 10.0 | | | | | |
| 39 | 0.77 | 10 | 1.0 | 1.1 | | | | | |
| 40 | 1.0 | 3.1 | 1.0 | 0.5 | | Yes | | | |

*Weak response observed without all animals in the group responding.
**Robust response with all animals in the group responding and some exhibiting multiple PEs.
¹NDP-α-MSH caused 22% decrease in ICV (1 nmole) food intake in 24 hrs.
²MT-II caused 54% decrease in ICV (1 nmole) food intake in 24 hrs.
³Functional antagonists known not to cause PE response.

Applicants have surprisingly and unexpectedly found that melanocortin receptor-specific compounds, which may be either peptide-based or small molecules, may be selected by the methods of this invention by determining intrinsic activity of the compound in both low and high density melanocortin receptor systems. In one aspect, compounds are selected which are useful for attenuating food intake and body weight gain generally such as for treatment of obesity and related energy homeostasis diseases, conditions and syndromes, which compounds do not induce a substantial, or any, sexual response in a mammal, such as an erectile response in a male. It has been recognized for several years that MC4-R agonists may be employed for the treatment of obesity and related energy homeostasis diseases, conditions and syndromes. However, a number of melanocortin-receptor specific compounds and agents heretofore evaluated for use in treatment of obesity and related conditions have had unacceptable side effects relating to initiation or induction of a sexual response, including a penile erection response in males. In part, compounds disclosed herein, and compounds selected by methods of the invention, do not generally induce any sexual response, and/or do not induce a substantial sexual response.

It has surprisingly and unexpectedly been discovered that compounds, which may be peptides, peptide-derived molecules, or small molecules, including small molecules with a ring core structure, which compounds have ascertainable intrinsic activity in a low density melanocortin receptor system, and specifically a low hMC4-R density system, will induce a sexual response in a mammal, such as an erectile response in a male, while compounds that do not have ascertainable intrinsic activity in a low density melanocortin receptor system, and specifically a low hMC4-R density system, will generally not induce a sexual response in a mammal, such as an erectile response in a male.

A first embodiment of the present invention provides a method for discriminating melanocortin receptor-specific compounds, and specifically MC4-R specific compounds, based upon an in vitro assay system, between those compounds likely to induce a sexual response and those likely not to induce a sexual response. Compounds that do not have an ascertainable intrinsic activity in a system with a low density of melanocortin receptors, and specifically a low density hMC4-R system, but which do have an ascertainable intrinsic activity in a high density melanocortin receptor system, and specifically a high hMC4-R density system, may be selected for use in attenuating food intake and body weight gain generally such as for use in the treatment of obesity, without inducing a substantial sexual response or any sexual response. The method of the first embodiment thus includes providing a low density melanocortin receptor system, and preferably a low density hMC4-R system, providing a high density melanocortin receptor system, preferably a high density hMC4-R system, testing a compound in each system, and selecting a compound for use in attenuating food intake and body weight gain generally such as for treatment of obesity without inducing a substantial or any sexual response by selecting a compound with no ascertainable intrinsic activity in the low density melanocortin receptor system but with ascertainable intrinsic activity in the high density melanocortin receptor system.

Compounds with an ascertainable intrinsic activity in the high density hMC4-R system have an intrinsic activity of greater than about 0.1 (10%), such as based on maximal stimulation of adenylyl cyclase achievable by the compound in the same high density hMC4-R system where the maximal stimulation achieved by α-MSH or NDP-α-MSH is designated as an intrinsic activity of 1.0 (100%). Preferably, such compounds have an intrinsic activity of greater than about 0.2 (20%), more preferably greater than about 0.3 (30%), still more preferably greater than about 0.5 (50%), and most preferably greater than about 0.7 (70%). Compounds that do not have an ascertainable intrinsic activity in a low density melanocortin receptor system, and specifically a low density hMC4-R system, have either no measurable intrinsic activity, or alternatively if the compounds have a measurable intrinsic activity, have an intrinsic activity of less than about 0.1 (10%), such as based on maximal stimulation of adenylyl cyclase achievable by the compound in the same low density hMC4-R system where the maximal stimulation achieved by α-MSH or NDP-α-MSH is designated as an intrinsic activity of 1.0 (100%).

The method disclosed above in the first embodiment may be used in combination with the methods disclosed in a related application entitled Compounds and Methods for Treatment of Obesity, U.S. patent application Ser. No. ______, filed concurrently herewith, Attorney Docket No. 056291-5365, and the specification thereof is incorporated herein by reference. The Compounds and Methods for Treatment of Obesity application discloses a method for selecting melanocortin receptor-specific compounds for treatment of obesity and related disorders, and in particular compounds for treatment of obesity and related energy homeostasis or feeding disorders characterized by excess weight gain, but which compounds do not induce a sexual response, including a penile response. In one embodiment of the methods of the Compounds and Methods for Treatment of Obesity application, a compound, which may be a peptide, peptide-derivative, or a small molecule, is selected which is an agonist or partial agonist as to MC4-R in a normal density melanocortin receptor system, but which may further be characterized in that the compound has high affinity, and on approximately the same order, as to MC4-R with respect to both α-MSH and AgRP, and thus attenuates the binding of both α-MSH and AgRP to MC4-R. This compound may further optionally be characterized in that it has a Ki (nM) at MC4-R, determined with respect to NDP-α-MSH, that is half or less than half, and preferably substantially less than half, of the EC₅₀ (nM) at MC4-R. Thus the EC₅₀ (nM) at MC4-R may be 2 times, 5 times, 10 times, 20 times, or more higher than the corresponding Ki (nM) at MC4-R, determined with respect to NDP-α-MSH. Thus, a compound may be selected on the basis of intrinsic activity in both a low and high density melanocortin receptor system, and may further, optionally and additionally be selected based upon one or more methods disclosed in the Compounds and Methods for Treatment of Obesity application, including the foregoing.

A second embodiment of the present invention provides a method for discriminating melanocortin receptor-specific compounds, and specifically MC4-R specific compounds, based upon an in vitro assay system, between those compounds likely to induce a sexual response and those likely not to induce a sexual response. Compounds that have an ascertainable intrinsic activity in a low density melanocortin receptor system, and specifically a low density hMC4-R system, and which further have an ascertainable intrinsic activity in a high density melanocortin receptor system, and specifically a high density hMC4-R system, may be selected for use in the treatment of sexual dysfunction, such as male erectile dysfunction and female sexual dysfunction. The method of the second embodiment thus includes providing a low density melanocortin receptor system, and preferably a low density hMC4-R system, providing a high density melanocortin receptor system, and preferably a high density hMC4-R system, testing a compound in each system, and selecting a compound for treatment of sexual dysfunction by selecting a compound with ascertainable intrinsic activity in the low density melanocortin receptor system and ascertainable intrinsic activity in the high density melanocortin receptor system.

Compounds with an ascertainable intrinsic activity in the low density hMC4-R system are characterized, in one aspect, by having an intrinsic activity of greater than about 0.1 (10%), based on maximal stimulation of adenylyl cyclase achievable by the compound in the same low density hMC4-R system where the maximal stimulation achieved by α-MSH or NDP-α-MSH is designated as an intrinsic activity of 1.0 (100%). Preferably such compounds have an intrinsic activity of greater than about 0.2 (20%), more preferably greater than about 0.3 (30%), still more preferably greater than about 0.5 (50%), and most preferably greater than about 0.7 (70%). Compounds with an ascertainable intrinsic activity in the high density hMC4-R system have an intrinsic activity of greater than about 0.1 (10%), based on maximal stimulation of adenylyl cyclase achievable by the compound in the same high density hMC4-R system where the maximal stimulation achieved by α-MSH or NDP-α-MSH is designated as an intrinsic activity of 1.0 (100%). Preferably such compounds have an intrinsic activity of greater than about 0.2 (20%), more preferably greater than about 0.3 (30%), still more preferably greater than about 0.5 (50%), and most preferably greater than about 0.7 (70%).

Preferably, such compounds selected for use in treatment of sexual dysfunction by the second embodiment described above can further be characterized in that such compounds are agonists as to MC4-R, which is to say, have an intrinsic activity of greater than about 0.7 (70%), such as based on maximal stimulation of adenylyl cyclase achievable by the compound in a normal density hMC4-R system where the maximal stimulation achieved by α-MSH or NDP-α-MSH is designated as an intrinsic activity of 1.0 (100%).

Compounds tested by the methods of the second embodiment are not likely to be effective for use in the treatment of sexual dysfunction, even if such compounds are agonists as to MC4-R as determined in a normal density hMC4-R system, if such compounds are inactive or have a low intrinsic activity, such as below about 0.1 (10%), preferably below about 0.3 (30%), based on maximal stimulation of adenylyl cyclase achievable by the compound in a normal density hMC4-R system where the maximal stimulation achieved by α-MSH or NDP-α-MSH is designated as an intrinsic activity of 1.0 (100%).

A third embodiment of the present invention provides a method for selecting neutral antagonist compounds that may be employed for treatment of energy homeostasis or feeding disorders or conditions and/or for attenuating food intake and weight gain generally. Such energy or feeding disorders and conditions include, but are not limited to, body weight disorders such as cachexia, sarcopenia and wasting syndrome or disease, and alternatively or additionally treatment of inflammation and immune disorders. Such energy or feeding disorders and conditions may further include treatment of obesity and related energy homeostasis or feeding disorders characterized by excess weight gain. Such compounds are further characterized in that they at least substantially do not induce and/or do not induce at all a sexual response in a mammal. Applicants have surprisingly and unexpectedly found that certain compounds which are classified as antagonists with respect to MC4-R in a normal density hMC4-R system, and thus by definition are not agonists or partial agonists as to as to MC4-R, are useful for attenuating food intake and weight gain generally such as for treatment of obesity and related energy homeostasis diseases, conditions and syndromes. Thus, the compounds of Examples 4, 12, 13, 23 and 26 of the Compounds and Methods for Treatment of Obesity application are classified as antagonists, with pA₂ values as given therein. The compounds of Examples 4, 12, 13, 23 and 26 of the Compounds and Methods for Treatment of Obesity application each caused a decrease in body weight under the described experimental conditions. Such neutral antagonist compounds may be selected as at least substantially not inducing a sexual response in a mammal, including at least substantially not inducing a penile erection in a male, if such compounds are inactive in both a low and high density melanocortin receptor system. Thus, such compounds have no ascertainable intrinsic activity in both a low and high density melanocortin receptor system, and specifically a low and high density hMC4-R system, where in each system the maximum measurable intrinsic activity, if any is less than about 0.1 (10%), based on maximal stimulation of adenylyl cyclase achievable by the compound in the same low or high density hMC4-R system where the maximal stimulation achieved by α-MSH or NDP-α-MSH is designated as an intrinsic activity of 1.0 (100%).

It may thus be seen that the invention, as described in the foregoing embodiments and elsewhere in this specification, provides new, novel and useful methods for selecting melanocortin receptor-specific compounds for modulating food intake and weight gain generally, such as for treating obesity and related energy homeostasis diseases, conditions and syndromes. Thus, the methods of the invention may be used to screen and select compounds with the desired pharmacological profiles. Heretofore, as described in the Background of the Invention, methods employed by others have simply provided for screening for agonist status, typically as to MC4-R, selecting agonist compounds, and testing such compounds. This invention illustrates that significantly different mechanisms of action are implicated in selection of compounds for the specific indication of obesity and related energy homeostasis diseases, conditions and syndromes, and further selection of compounds which do not cause unintended effects, such as inducing a sexual response.

The invention, in part, relates to the discovery that determining the efficacy of specific compounds in terms of cAMP expression in a low density melanocortin receptor system, and specifically a low density hMC4-R system, and in terms of a high density melanocortin receptor system, and specifically a high density hMC4-R system, provides valuable and specific information relating directly to the potential clinical utility of such compounds. This thus permits compounds to readily be identified and categorized as compounds with potential utility for attenuating food intake and weight gain generally such as for treatment of obesity and related energy homeostasis diseases, at least substantially without causing sexual responses, and conversely permits compounds to readily be identified and categorized as compounds for treatment of sexual dysfunction, including male erectile dysfunction and female sexual disorder.

It may be further be seen that the invention provides methods that may readily be adapted for use to screen, select and characterize any melanocortin receptor-specific compound, whether a peptide, peptide derivative, small molecule or otherwise. It may further be seen that the methods may be adapted, by selecting different reactants, reaction conditions, reagents, or the like, and still remain within the scope of this invention. Thus, by way of example, and not limitation, other reference compounds may be used in place of α-MSH, such as other known or hereafter development melanocortin agonists, other variable gene expression regulatory systems may be employed, other vectors and genetic constructs may be employed, other cell lines may be employed, and the like. In particular, other means and methods for determining the functional activity of a compound, including but not limited to the means and methods heretofore described, may be employed. In general, once the principles of the invention are understood, one of ordinary skill in the art can determine and develop alternative means and methods of practicing the invention. It is intended to include all such variations within the invention.

While the invention is exemplified in terms of differentiation of the effects of melanocortin receptor-specific agents between effects associated with attenuation of food intake and/or body weight and those effects associated with a sexual response, including a penile erection response in males, it may readily be seen that the methods of the invention may be applied to other effects and responses associated with or resulting from administration of a melanocortin receptor-specific agent. In particular, systemic effects resulting from administration of a melanocortin receptor-specific agent, particularly an agonist agent, such as pressor effects resulting in an increase in blood pressure, pica behavior, disruption of normal behavioral satiety sequences and various other systemic effects, are believed to be associated with potent agonist responses derived through central melanocortin agents, and are thus believed to be associated with the mechanisms resulting in a sexual response, and not with the mechanisms associated with attenuation of food intake and/or body weight. Thus in another aspect the invention provides a method for differentiation and selection of melanocortin receptor-specific agents useful for attenuating food intake but which agents do not have, or do not substantially have, a pressor effect or other effects associated with potent agonist responses or, in particular, agonist responses or intrinsic activity in a low density melanocortin receptor system, and in particular a low density MC4-R system.

In each of the following examples, the data presented in Table 3 is incorporated by reference, including specifically the data on “Efficacy/Intrinsic Activity” and “EC₅₀ (nM)” in both the low density hMCR-4 density system (0.1 ng/ML doxycycline) and the high density hMCR-4 density system (10 ng/ML doxycycline). The invention is illustrated by the following non-limiting examples, it being understood that similar or related methods may be employed with different compounds, and yet be within the scope of the invention.

### Example 1

### NH₂—(CH₂)₆—C(═O)-Ser(Bzl)-(D)Phe(4-Cl)-Arg-Trp-NH₂

The peptide compound NH₂—(CH₂)₆—C(═O)-Ser(Bzl)-(D)Phe(4-Cl)-Arg-Trp-NH₂ was synthesized by peptide synthesis methods as disclosed in International Patent Application No. PCT/US02/22196. The molecular weight was determined to be 845. Competitive inhibition testing of the compound yielded the following results:

| | | | |
|---|---|---|---|
| MC1-R | MC3-R | MC4-R | MC5-R |

Inhibition at 1 μM (NDP-α-MSH) In Normal System

| | | | |
|---|---|---|---|
| 33% | 94% | 99% | 73% |

Ki (nM) (NDP-α-MSH) In Normal System

| | | | |
|---|---|---|---|
| 351 | 27 | 3 | 208 |

| Low Receptor Density System | | | |
|---|---|---|---|
| Efficacy/Intrinsic | | High Receptor Density System | |
| Activity | EC₅₀ (nM) | Efficacy/Intrinsic Activity | EC₅₀ (nM) |
| None | N/A | 0.97 | 21 |

In a cAMP assay using MC1-R, MC4-R and MC5-R, at 1 μM concentrations the compound of Example 1 was a partial agonist at MC4-R.

The EC₅₀ (nM) at MC4-R in a normal system was determined to be 296 with intrinsic activity of 0.32.

In rat model IV feeding studies at 0.75 mg/kg dose levels, a maximal 37% decrease was observed in food intake for a period of 24 hours. In rat model ICV feeding studies at 1 nmol dose levels, a maximal 21% decrease was observed in food intake for a period of 24 hours. In mouse model IP feeding studies at 3 mg/kg dose levels, a maximal 26% decrease was observed in food intake for a period of 4 hours. In mouse model IN feeding studies at 10 μg/kg dose levels, a maximal 16% decrease was observed in food intake for a period of 4 hours.

In rat model penile behavior induction experiments by ICV administration of doses from 0.01 to 5 nmol, no penile erection response was observed.

### Example 2

### N-(3-{(S)-1-[(R)-2-Amino-3-(2,4-dichloro-phenyl)-propionyl]-4-[2-(1H-indol-3-yl)-ethyl]-3-oxo-piperazin-2-yl}-propyl)-guanidine

A compound of the following structure:

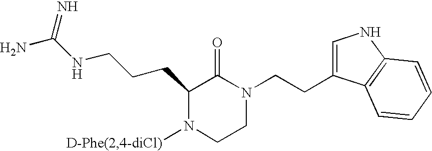

was synthesized by methods described in U.S. patent application Ser. No. 10/762,079. The molecular weight was determined to be 557.5 ESI-MS (M+1). Competitive inhibition testing of the compound yielded the following results (average of triplicates with actual mean values described):

| | | | |
|---|---|---|---|
| MC1-R | MC3-R | MC4-R | MC5-R |

Inhibition at 1 μM (NDP-α-MSH) In Normal System

| | | | |
|---|---|---|---|
| 7% | 57% | 96% | 35% |

Ki (nM) (NDP-α-MSH) In Normal System

| | | | |
|---|---|---|---|
| 1409 | 533 | 15 | 1578 |

| Low Receptor Density System | | | |
|---|---|---|---|
| Efficacy/Intrinsic | | High Receptor Density System | |
| Activity | EC₅₀ (nM) | Efficacy/Intrinsic Activity | EC₅₀ (nM) |
| None | N/A | 0.56 | 57 |

In a cAMP assay using MC1-R, MC4-R and MC5-R, at 1 μM concentrations the compound of Example 2 was a partial agonist at MC4-R, MC5-R and MC1-R.

The EC₅₀ (nM) at MC4-R in a normal system was determined to be 604 with intrinsic activity of 0.3.

In rat model penile behavior induction experiments at 0.3 to 3 μg/kg (IV), the compound did not cause penile erection behavior.

### Example 3

### N-{3-[1-[2(R)-Amino-3-(4-chloro-2-fluoro-phenyl)-propionyl]-5(S)-methyl-4-(2-naphthalen-2-yl-ethyl)-piperazin-2(S)-yl]-propyl}-guanidine

The following compound was synthesized by methods described in U.S. patent application Ser. No. 10/837,519, using 2-naphthylacetic acid as J-COOH, D-alanine methyl ester as NH₂—CH(R₅)—COOCH₃, Fmoc-Arg(Boc)₂-OH as Prt-NH—CH(R₂)—COOH, and Boc-D-2-fluoro,4-chloro-Phe-OH as Q-COOH. It was tested as described above with the results shown. The mass was analyzed as 553 (M+H).

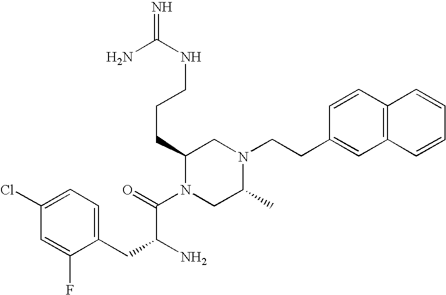

Inhibition at 1 μM (NDP-α-MSH) In Normal System

| MC1-R | MC3-R | MC4-R | MC5-R |
|---|---|---|---|
| 57% | 82% | 99% | 59% |

Ki (nM) (NDP-α-MSH) In Normal System

| MC1-R | MC3-R | MC4-R | MC5-R |
|---|---|---|---|
| 109 | 186 | 6 | 204 |
| Low Receptor | | High Receptor | |
| Density System | | Density System | |
| Efficacy/Intrinsic | | Efficacy/Intrinsic | |
| Activity | EC₅₀ (nM) | Activity | EC₅₀ (nM) |
| None | N/A | 0.26 | 61 |

In a cAMP assay using MC1-R, MC4-R and MC5-R, at 1 μM concentrations the compound of Example 3 exhibited no intrinsic activity (inactive) at MC4-R and MC5-R and was a partial agonist at MC1-R.

### Example 4

### N-{3-[1-[2(R)-Amino-3-(2,4-dichloro-phenyl)-propionyl]-5(R)-isobutyl-4-(2-naphthalen-2-yl-ethyl)-piperazin-2(S)-yl]-propyl}-guanidine

The following compound was synthesized by the methods of both Schemes 3 and 5 described in U.S. patent application Ser. No. 10/837,519, using 2-naphthylacetic acid as J-COOH, D-Leucinol as NH₂—CH(R₅)—CH(R₄)—OH, D-leucine methyl ester as NH₂—CH(R₅)—COOCH₃, Fmoc-L-Arg(Boc)₂-OH as Prt-NH—CH(R₂)—COOH, and Boc-D-2,4-dichloro-Phe-OH as Q-COOH. It was tested as described above with the results shown. The mass was analyzed as 611.1 (M+H).

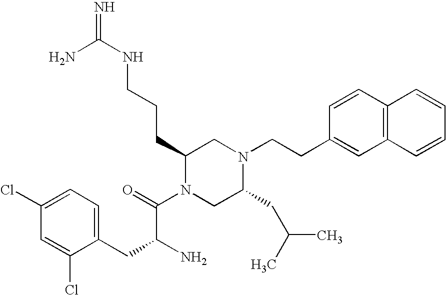

Inhibition at 1 μM (NDP-α-MSH) In Normal System

| MC1-R | MC3-R | MC4-R | MC5-R |
|---|---|---|---|
| 21% | 64% | 99% | 75% |

Ki (nM) (NDP-α-MSH) In Normal System

| MC1-R | MC3-R | MC4-R | MC5-R |
|---|---|---|---|
| 1364 | 87 | 5 | 160 |
| Low Receptor | | High Receptor | |
| Density System | | Density System | |
| Efficacy/Intrinsic | | Efficacy/Intrinsic | |
| Activity | EC₅₀ (nM) | Activity | EC₅₀ (nM) |
| None | N/A | None | N/A |

In a cAMP assay using MC1-R, MC4-R and MC5-R, at 1 μM concentrations the compound of Example 4 exhibited no intrinsic activity at MC1-R and MC4-R, and was a partial agonist at MC5-R. The compounds tested as antagonist as to MC4-R in a normal system with a pA₂ value of 7.7.

In mouse model IP feeding studies at 10 mg/kg dose levels, a maximal 60% decrease was observed in food intake for a period of 4 hours.

### Example 5

### N-{3-[1-(2(R)-Amino-3-naphthalen-2-yl-propionyl)-5(R)-methyl-4-(2-p-tolyl-ethyl)-piperazin-2(S)-yl]-propyl}-guanidine

The following compound was synthesized by the method of Scheme 6 in U.S. patent application Ser. No. 10/837,519 using 4-methylphenylacetaldhyde as J-aldehyde, D-alanine methyl ester as NH₂—CH(R₅)—COOCH₃, and Boc-D-2-Nal-OH as Q-COOH. It was tested as described above with the results shown. The mass was analyzed as 515.4 (M+H).

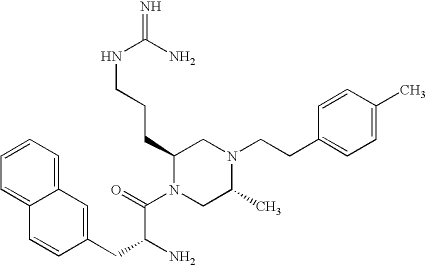

Inhibition at 1 μM (NDP-α-MSH) In Normal System

| MC1-R | MC3-R | MC4-R | MC5-R |
|---|---|---|---|
| 32% | 61% | 95% | 65% |

Ki (nM) (NDP-α-MSH) In Normal System

| MC1-R | MC3-R | MC4-R | MC5-R |
|---|---|---|---|
| 135 | 164 | 29 | 448 |
| Low Receptor | | High Receptor | |
| Density System | | Density System | |
| Efficacy/Intrinsic | | Efficacy/Intrinsic | |
| Activity | EC₅₀ (nM) | Activity | EC₅₀ (nM) |
| None | N/A | 0.46 | 117 |

In a cAMP assay using MC1-R, MC3-R, MC4-R and MC5-R, at 1 μM concentrations the compound of Example 5 exhibited no intrinsic activity at MC3-R, and was a partial agonist at MC1-R, MC4-R and MC5-R. An EC₅₀ value of 370 nM on MC-4R in a normal system was determined with an intrinsic activity of 0.6.

In rat model IV penile erection induction experiments at doses ranging from 0.3 to 30 μg/kg no penile erection response was observed.

### Example 6

### N-{3-[1-(2(R)-Amino-3-naphthalen-2-yl-propionyl)-4-(2-1H-indol-3-yl-acetyl)-5(R)-methyl-piperazin-2(S)-yl]-propyl}-guanidine

The following compound was synthesized by the method of Scheme 6 in U.S. patent application Ser. No. 10/837,519 using indole-3-acetic acid as J-COOH, D-alanine methyl ester as NH₂—CH(R₅)—COOCH₃, and Boc-D-2-Nal-OH as Q-COOH. It was tested as described above with the results shown. The mass was analyzed as 554.4 (M+H).

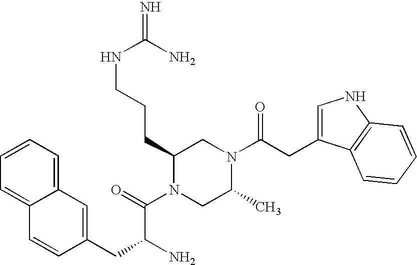

Inhibition at 1 μM (NDP-α-MSH) In Normal System

| MC1-R | MC3-R | MC4-R | MC5-R |
|---|---|---|---|
| 20% | 35% | 90% | 6% |

Ki (nM) (NDP-α-MSH) In Normal System

| MC1-R | MC3-R | MC4-R | MC5-R |
|---|---|---|---|
| 315 | 1822 | 35 | 1527 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | 0.43 | 39 |

In a cAMP assay using MC1-R, MC3-R, MC4-R and MC5-R, at 1 μM concentrations the compound of Example 6 exhibited no intrinsic activity at MC1-R and MC3-R, and was a partial agonist at MC4-R and MC5-R. The EC₅₀ value for MC4-R in a normal system was over 1000 nM with an intrinsic activity of 0.2.

In rat model IV penile erection induction experiments at doses ranging from 0.3 to 30 μg/kg no penile erection response was observed.

### Example 7

### N-{3-[1-(2(R)-Amino-3-naphthalen-2-yl-propionyl)-4-(2-1H-indol-3-yl-butyryl)-5(R)-methyl-piperazin-2(S)-yl]-propyl}-guanidine

The following compound was synthesized by the method of Scheme 6 in U.S. patent application Ser. No. 10/837,519 using indole-3-butyric acid as J-COOH, D-alanine methyl ester as NH₂—CH(R₅)—COOCH₃, and Boc-D-2-Nal-OH as Q-COOH. It was tested as described above with the results shown. The mass was analyzed as 582.6 (M+H).

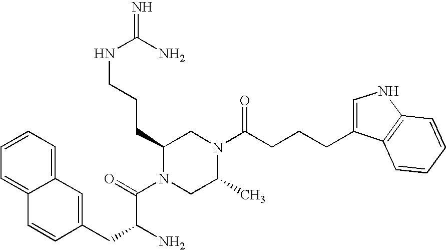

Inhibition at 1 μM (NDP-α-MSH) In Normal System

| MC1-R | MC3-R | MC4-R | MC5-R |
|---|---|---|---|
| 0% | 32% | 88% | 62% |

Ki (nM) (NDP-α-MSH) In Normal System

| MC1-R | MC3-R | MC4-R | MC5-R |
|---|---|---|---|
| 1203 | 657 | 90 | 271 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | 0.38 | 72 |

In a cAMP assay using MC1-R, MC3-R, MC4-R and MC5-R, at 1 μM concentrations the compound of Example 7 exhibited no intrinsic activity at MC1-R, MC3-R and MC5-R, and was a partial agonist at MC4-R. The EC₅₀ value for MC4-R in a normal system was over 3000 nM with an intrinsic activity of 0.4.

In rat model penile behavior induction experiments by ICV administration of doses from 0.01 to 10 nmol, no penile erection response was observed.

### Example 8

### N-(3-{1-(2(R)-Amino-3-naphthalen-2-yl-propionyl)-5(R)-methyl-4-[2-(2-methyl-1H-indol-3-yl)-ethyl]-piperazin-2(S)-yl}-propyl)-guanidine

The following compound was synthesized by the method of Scheme 6 in U.S. patent application Ser. No. 10/837,519 using 2-methyl-indole-3-acetaldehyde as J-aldehyde, D-alanine methyl ester as NH₂—CH(R₅)—COOCH₃, and Boc-D-2-Nal-OH as Q-COOH. It was tested as described above with the results shown. The mass was analyzed as 554.5 (M+H).

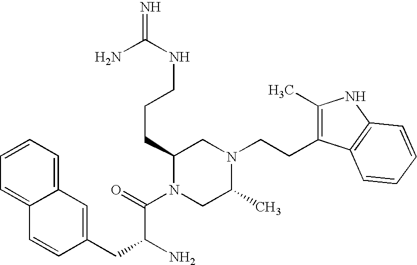

Inhibition at 1 μM (NDP-α-MSH) In Normal System

| MC1-R | MC3-R | MC4-R | MC5-R |
|---|---|---|---|
| 73% | 57% | 95% | 76% |

Ki (nM) (NDP-α-MSH) In Normal System

| MC1-R | MC3-R | MC4-R | MC5-R |
|---|---|---|---|
| 148 | 451 | 26 | 293 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | None | N/A |

In a cAMP assay using MC1-R, MC3-R, MC4-R and MC5-R, at 1 μM concentrations the compound of Example 8 exhibited no intrinsic activity at MC3-R, was a partial agonist at MC1-R, and was an agonist at MC4-R and MC5-R. The EC₅₀ value for MC4-R in a normal system was over 384 nM with an intrinsic activity of 0.9.

In rat model penile behavior induction experiments by ICV administration of doses from 0.01 to 10 nM, no penile erection response was observed.

### Example 9

### N-(3-{1-(2(R)-Amino-(4-chloro-2-methyl-phenyl)-propionyl)-5(R)-methyl-4-[2-(1H-indol-3-yl)-ethyl]-piperazin-2(S)-yl}-propyl)-guanidine

The following compound was synthesized by the method of Scheme 5 in U.S. patent application Ser. No. 10/837,519 using indole-3-acetic acid as J-COOH, D-alanine methyl ester as NH₂—CH(R₅)—COOCH₃, and Boc-D-4-chloro-2-methyl-Phe-OH as Q-COOH. It was tested as described above with the results shown. The mass was analyzed as 538.6 (M+H).

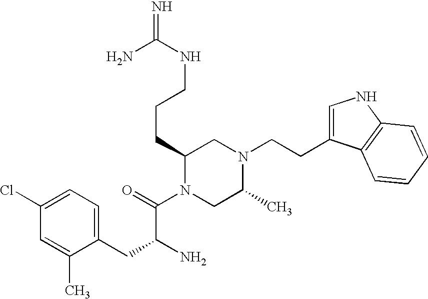

Inhibition at 1 μM (NDP-α-MSH) In Normal System

| MC1-R | MC3-R | MC4-R | MC5-R |
|---|---|---|---|
| 40% | 74% | 98% | 75% |

Ki (nM) (NDP-α-MSH) In Normal System

| MC1-R | MC3-R | MC4-R | MC5-R |
|---|---|---|---|
| 1730 | 177 | 5 | 360 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | 0.73 | 22 |

In a cAMP assay using MC1-R, MC3-R, MC4-R and MC5-R, at 1 μM concentrations the compound of Example 9 exhibited no intrinsic activity at MC1-R, MC3-R and MC5-R, and was a full agonist at MC4-R. The EC₅₀ value for MC4-R in a normal system was over 7 nM with an intrinsic activity of 1.

In rat model ICV feeding studies at 1 nmol dose levels, a 24 hour decrease in food intake of 16%, and decrease in body weight of 2%, was observed.

In mouse model IP feeding studies at 3 mg/kg dose levels, a maximal 10% decrease was observed in food intake for a period of 4 hours.

In rat model IV and ICV penile erection induction experiments at doses ranging from 0.3 to 30 μg/kg given IV and at 0.01 to 10 nmole given ICV, no penile erection response was observed.

### Example 10

### N-{3-[1-[2(R)-Amino-3-(4-chloro-2-dimethyl-phenyl)-propionyl]-5(R)-methyl(2-naphthalen-2-yl-acetyl)-piperazin-2(S)-yl]-propyl}-guanidine

The following compound was synthesized by the method of Scheme 7 in U.S. patent application Ser. No. 10/837,519 using 2-naphthyl acetic acid as J-COOH, L-Orn(Boc) methyl ester as NH₂—CH(R₂)—COOCH₃, and Boc-D-4-chloro-2-methyl-Phe-OH as Q-COOH. It was tested as described above with the results shown. The mass was analyzed as 563.4 (M+H).

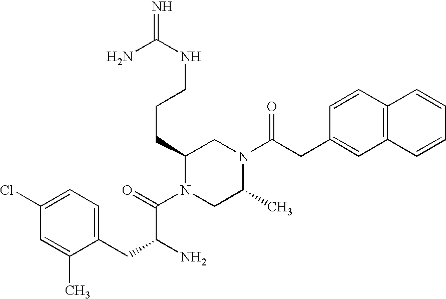

Inhibition at 1 μM (NDP-α-MSH) In Normal System

| MC1-R | MC3-R | MC4-R | MC5-R |
|---|---|---|---|
| 3% | 34% | 96% | 36% |

Ki (nM) (NDP-α-MSH) In Normal System

| MC1-R | MC3-R | MC4-R | MC5-R |
|---|---|---|---|
| 462 | 398 | 3 | 774 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| 0.23 | 38 | 0.69 | 26 |

In a cAMP assay for determination of agonist/antagonist status, it was determined that the compound was a partial agonist as to MC1-R and MC3-R, and an agonist as to MC4-R and MC5-R. The EC₅₀ value for MC4-R in a normal system was 18 nM with an intrinsic activity of 0.9.

In rat model IV feeding studies at 10 mg/kg dose levels, a maximal 33% decrease was observed in food intake for a period of 24 hours.

In mouse model IP feeding studies at 10 mg/kg dose levels, a maximal 27% decrease was observed in food intake for a period of 4 hours.

In rat model IV penile erection induction experiments at doses ranging from 0.3 to 30 μg/kg, 0.5 to 0.7 mean penile erections per rat were observed with not more than 66% animals responding.

### Example 11

### N-{3-[1-[2(R)-Amino-3-(2,4-dichloro-phenyl)-propionyl]-5(R)-methyl-4-(2-naphthalen-2-yl-ethyl)-piperazin-2(S)-yl]-propyl}-guanidine

The following compound was synthesized by the methods of both Schemes 3 and 5 in U.S. patent application Ser. No. 10/837,519 using 2-naphthylacetic acid as J-COOH, (R)-(−)-2-amino-1-propanol as NH₂—CH(R₅)—CH(R₄)—OH, Fmoc-L-Arg(Boc)₂-OH as Prt-NH—C(R₂)—COOH, D-alanine methyl ester as NH₂—CH(R₅)—COOCH₃ and Boc-D-2,4-dichloro-Phe-OH as Q-COOH. It was tested as described above with the results shown. The mass was analyzed as 569.3 (M+H).

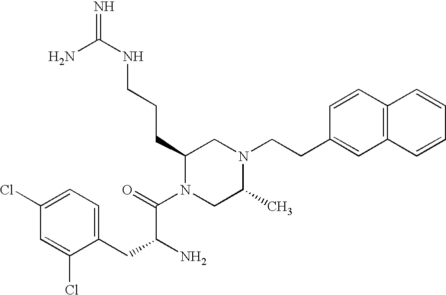

Inhibition at 1 μM (NDP-α-MSH) In Normal System

| MC1-R | MC3-R | MC4-R | MC5-R |
|---|---|---|---|
| 20% | 72% | 99% | 65% |

Ki (nM) (NDP-α-MSH) In Normal System

| MC1-R | MC3-R | MC4-R | MC5-R |
|---|---|---|---|
| 1134 | 95 | 2 | 362 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | 0.49 | 61 |

In a cAMP assay for determination of agonist/antagonist status, it was determined that the compound was an antagonist as to MC4-R with a pA₂ value in a normal system of 7.8. At a 1 μM concentration it was inactive at MC1-R, MC3-R and MC5-R.

In rat model IV and IP feeding studies at 10 mg/kg dose levels, a maximal 87% (IV) and 19% (IP) decrease was observed in food intake for a period of 24 hours.

In mouse model IP feeding studies at 3 and 10 mg/kg dose levels, a maximal 10% and 30% decrease, respectively was observed in food intake for a period of 4 hours.

### Example 12

### N-{2-[2(S)-(3-Guanidino-propyl)-5(R)-isobutyl-4-(2-naphthalen-2-yl-ethyl)-piperazin-1-yl]-1(R)-naphthalen-2-ylmethyl-2-oxo-ethyl}-acetamide

The following compound was synthesized by the method of Scheme 5 in U.S. patent application Ser. No. 10/837,519 using 2-naphthylacetic acid as J-COOH, D-leucinol as NH₂—CH(R₅)—CH(R₄)—OH, D-leucine methyl ester as NH₂—CH(R₅)—COOCH₃, and Boc-D-2-Nal-OH as Q-COOH as described in Example 35 thereof. An acetyl group was attached to the amino group of D-2-Nal by reaction of the compound of Example 35 with Ac-OSu in DMF. It was tested as described above with the results shown. The mass was analyzed as 635.9 (M+H).

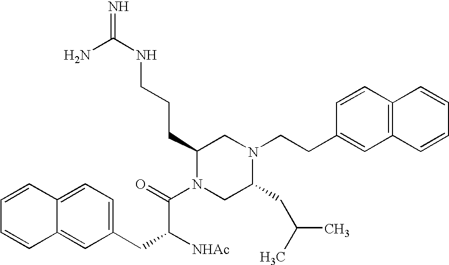

Inhibition at 1 μM (NDP-α-MSH) In Normal System

| MC1-R | MC3-R | MC4-R | MC5-R |
|---|---|---|---|
| 0% | 40% | 100% | 64% |

Ki (nM) (NDP-α-MSH) In Normal System

| MC1-R | MC3-R | MC4-R | MC5-R |
|---|---|---|---|
| 4616 | 474 | 6 | 378 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | None | N/A |

In a cAMP assay using MC1-R, MC3-R, MC4-R and MC5-R, at 1 μM concentrations the compound of Example 13 exhibited no intrinsic activity at MC1-R, MC3-R and MC4-R, and was a partial agonist at MC5-R. At MC4-R in a normal system it tested as an antagonist with a pA₂ value of 7.3.

In mice model IP feeding studies at 10 mg/kg dose levels, a maximal 46% decrease was observed in food intake for a period of 4 hours.

### Example 13

### NH₂—(CH₂)₆—C(═O)-Ser(Bzl)-D-Phe(4-Cl)-Arg-Trp-Asp-NH₂

The peptide compound NH₂—(CH₂)₆—C(═O)-Ser(Bzl)-D-Phe(4-Cl)-Arg-Trp-Asp-NH₂ was synthesized by standard peptide synthesis methods as described in International Patent Application No. PCT/US02/22196. It was tested as described above with the results shown.

| | | | |
|---|---|---|---|
| MC1-R | MC3-R | MC4-R | MC5-R |

Inhibition at 1 μM (NDP-α-MSH) In Normal System

| | | | |
|---|---|---|---|
| 35% | 73% | 98% | 48% |

Ki (nM) (NDP-α-MSH) In Normal System

| | | | |
|---|---|---|---|
| 760 | 180 | 9 | 596 |

| Low Receptor Density System | | | |
|---|---|---|---|
| Efficacy/Intrinsic | | High Receptor Density System | |
| Activity | EC₅₀ (nM) | Efficacy/Intrinsic Activity | EC₅₀ (nM) |
| None | N/A | 0.72 | 34 |

In a cAMP assay for determination of functionality as to MC4-R, it was determined that the compound in a normal system had an EC₅₀ (nM) of 568, showed intrinsic activity of 0.5, and was a partial agonist as to MC4-R. It was inactive at MC1-R and MC5-R and a partial agonist at MC3-R.

In rat model ICV feeding studies at 1 nmol dose levels, a 24 hour decrease in food intake of 25.7%, and decrease in body weight of 2.1%, was observed. In IV feeding studies, a 24 hour decrease in food intake of 6%, and decrease in body weight of 1%, was observed.

In rat model IV penile erection induction experiments at 1 μPK, no penile erections were observed.

### Example 14

### NH₂—(CH₂)₆—C(═O)-Ser(Bzl)-D-Phe(4-Cl)-Arg-Trp-Asp-Phe-NH₂

The peptide corn pound NH₂—(CH₂)₆—C(═O)-Ser(Bzl)-D-Phe(4-Cl)-Arg-Trp-Asp-Phe-NH₂ was synthesized by standard peptide synthesis methods as described in International Patent Application No. PCT/US02/22196. It was tested as described above with the results shown.

| | | | |
|---|---|---|---|
| MC1-R | MC3-R | MC4-R | MC5-R |

Inhibition at 1 Mm (NDP-α-MSH) In Normal System

| | | | |
|---|---|---|---|
| 38% | 66% | 99% | 52% |

Ki (nM) (NDP-α-MSH) In Normal System

| | | | |
|---|---|---|---|
| 242 | 76 | 5 | 405 |

| Low Receptor Density System | | | |
|---|---|---|---|
| Efficacy/Intrinsic | | High Receptor Density System | |
| Activity | EC₅₀ (nM) | Efficacy/Intrinsic Activity | EC₅₀ (nM) |
| None | N/A | 0.64 | 141 |

In a cAMP assay for determination of functionality as to MC4-R, it was determined that the compound was a partial agonist as to MC4-R. The EC₅₀ for MC4-R in a normal system was 238 nM and showed intrinsic activity of 0.5.

In rat model ICV feeding studies at 1 nmol dose levels, a 24 hour decrease in food intake of 50%, and decrease in body weight of −2%, was observed. In rat model IV feeding studies, a 24 hour decrease in food intake of 30%, and decrease in body weight of 2%, was observed.

In rat model IV penile erection induction experiments at 1 μPK, no penile erection response was observed.

### Example 15

### NH₂—(CH₂)₆—C(═O)-Ser(Bzl)-D-Phe(4-Cl)-Arg-Trp-Ala-NH₂

The peptide compound NH₂—(CH₂)₆—C(═O)-Ser(Bzl)-D-Phe(4-Cl)-Arg-Trp-Ala-NH₂ was synthesized by standard peptide synthesis methods as described in International Patent Application No. PCT/US02/22196. It was tested as described above with the results shown.

| | | | |
|---|---|---|---|
| MC1-R | MC3-R | MC4-R | MC5-R |

Inhibition at 1 μM (NDP-α-MSH) In Normal System

| | | | |
|---|---|---|---|
| 60% | 93% | 100% | 79% |

Ki (nM) (NDP-α-MSH) In Normal System

| | | | |
|---|---|---|---|
| 142 | 35 | 2 | 112 |

| Low Receptor Density System | | | |
|---|---|---|---|
| Efficacy/Intrinsic | | High Receptor Density System | |
| Activity | EC₅₀ (nM) | Efficacy/Intrinsic Activity | EC₅₀ (nM) |
| None | N/A | 0.67 | 55 |

In a cAMP assay for determination of functionality as to MC4-R, it was determined that the compound had in a normal system an EC₅₀ (nM) of >1 μm, showed intrinsic activity of less than 0.1, and was functionally inactive as to MC4-R.

In rat model ICV feeding studies at 1 nmol dose levels, a 24 hour decrease in food intake of 14%, and decrease in body weight of 2%, was observed. In rat model IV feeding studies, a 24 hour decrease in food intake of 4% was observed.

In rat model IV penile erection induction experiments at 1 μPK, no penile erection response was observed.

### Example 16

### 7′-amino-heptanoyl-Ser(Bzl)-D-Phe(4-Cl)-Arg-S-(−)-1-(1-naphthyl)ethylamide

The following peptide compound was synthesized by standard peptide synthesis methods as described in International Patent Application No. PCT/US02/22196. It was tested as described above with the results shown.

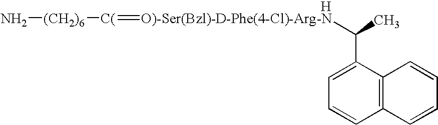

Inhibition at 1 μM (NDP-α-MSH) In Normal System

| MC1-R | MC3-R | MC4-R | MC5-R |
|---|---|---|---|
| 46% | 85% | 98% | 64% |

Ki (nM) (NDP-α-MSH) In Normal System

| MC1-R | MC3-R | MC4-R | MC5-R |
|---|---|---|---|
| 338 | 125 | 7 | 313 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | 0.63 | 180 |

In a cAMP assay for determination of functionality as to MC4-R, it was determined that the compound had in a normal system an EC₅₀ (nM) of 17, showed intrinsic activity of 0.3, and was a partial agonist as to MC4-R.

In rat model ICV feeding studies at 1 nmol dose levels, a 24 hour decrease in food intake of 13%, and decrease in body weight of 1%, was observed.

In rat model IV penile erection induction experiments at a dose of 1 μg/kg no penile erection response was observed.

### Example 17

### N-{3-[(S)-1-[(R)-2-Amino-3-(2,4-dichloro-phenyl)-propionyl]-4-(2-naphthalen-2-yl-ethyl)-piperazin-2-yl]-propyl}-guanidine

The following compound was synthesized by methods described in U.S. patent application Ser. No. 10/762,079. It was tested as described above with the results shown.

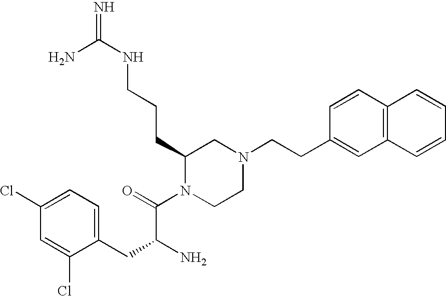

Inhibition at 1 μM (NDP-α-MSH) In Normal System

| MC1-R | MC3-R | MC4-R | MC5-R |
|---|---|---|---|
| 6% | 87% | 99% | 75% |

Ki (nM) (NDP-α-MSH) In Normal System

| MC1-R | MC3-R | MC4-R | MC5-R |
|---|---|---|---|
| 1198 | 97 | 3 | 259 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | 0.67 | 847 |

In a cAMP assay for determination of functionality as to MC4-R, it was determined that the compound had in a normal system an EC₅₀ (nM) of 22, showed intrinsic activity of 0.4, and was a partial agonist as to MC4-R.

In rat model IV feeding studies at 10 mg/kg dose levels, a maximal 91% decrease was observed in food intake for a period of 24 hours. In rat model ICV feeding studies at 1 nmole dose level, a maximal 32% decrease was observed in food intake for a period of 24 hours.

In mouse model IP feeding studies at 10 mg/kg dose levels, a maximal 86% decrease was observed in food intake for a period of 4 hours.

In rat model IV penile erection induction experiments at doses ranging from 0.3 to 30 μg/kg no penile erection response was observed. In rat model ICV penile erection induction experiments at doses ranging from 0.3 to 3 nmole no penile erection response was observed.

### Example 18

### (2S,5R)-5-phenyl-pyrrolidine-2-carboxylic acid [(R)-2-[(S)-2-(4-amino-butyl)-4-(2-naphthalen-2-yl-ethyl)-3-oxo-piperazin-1-yl]-1-(2,4-dichloro-benzyl)-2-oxo-ethyl]-amide

The following compound was synthesized. It was tested as described above with the results shown.

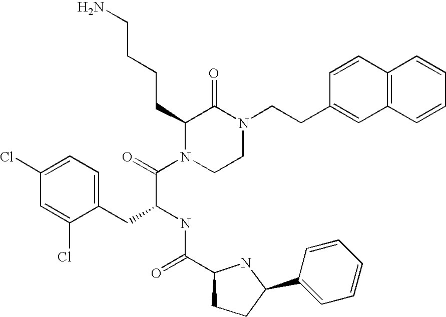

Inhibition at 1 μM (NDP-α-MSH) In Normal System

| MC1-R | MC3-R | MC4-R | MC5-R |
|---|---|---|---|
| 0% | 18% | 92% | 51% |

Ki (nM) (NDP-α-MSH) In Normal System

| MC1-R | MC3-R | MC4-R | MC5-R |
|---|---|---|---|
| 1370 | 855 | 50 | 789 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | 0.29 | 281 |

In a cAMP assay for determination of functionality as to MC4-R, it was determined that the compound had in a normal system an EC₅₀ (nM) of 959, showed intrinsic activity of 0.2, and was a partial agonist as to MC4-R.

In rat model ICV feeding studies at 1 nmol dose levels, a 24 hour decrease in food intake of 31%, and decrease in body weight of 2%, was observed.

### Example 19

### (2S,5R)-5-Phenyl-pyrrolidine-2-carboxylic acid {(R)-1-(2,4-dichloro-benzyl)-2-[(2S,5R)-2-(3-guanidino-propyl)-5-(naphthalen-2-yloxymethyl)-3-oxo-hexahydro-pyrrolo[1,2-a]imidazol-1-yl]-2-oxo-ethyl}-amide

The following compound was synthesized. It was tested as described above with the results shown.

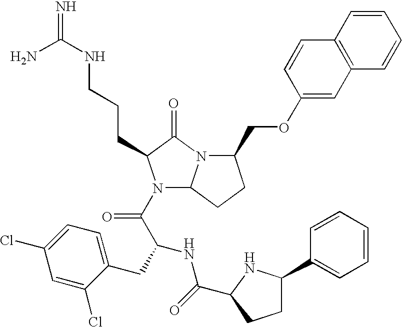

Inhibition at 1 μM (NDP-α-MSH) In Normal System

| MC1-R | MC3-R | MC4-R | MC5-R |
|---|---|---|---|
| 16% | 43% | 97% | 86% |

Ki (nM) (NDP-α-MSH) In Normal System

| MC1-R | MC3-R | MC4-R | MC5-R |
|---|---|---|---|
| 1041 | 312 | 12 | 99 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | 0.22 | 173 |

In a cAMP assay for determination of functionality as to MC4-R, it was determined that the compound had no intrinsic activity.

In rat model ICV feeding studies at 1 nmol dose levels, a 24 hour decrease in food intake of 38%, and decrease in body weight of 3%, was observed. In rat model IV feeding studies, a 24 hour decrease in food intake of 1%, and decrease in body weight of 2%, was observed.

In rat model IV penile erection induction experiments at a dose of 1 μg/kg no penile erection response was observed.

### Example 20

### (E)-N—[(R)-2-[(S)-2-(4-Amino-butyl)-4-(2-naphthalen-2-yl-ethyl)-3-oxo-piperazin-1-yl]-1-(2,4-dichloro-benzyl)-2-oxo-ethyl]-3-phenyl-acrylamide

The following compound was synthesized. It was tested as described above with the results shown.

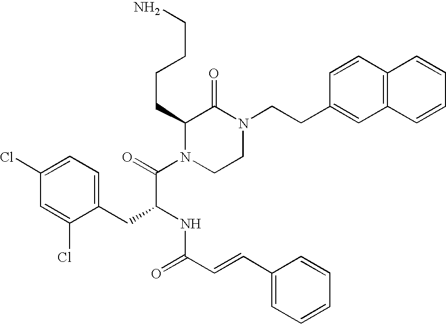

Inhibition at 1 μM (NDP-α-MSH) In Normal System

| MC1-R | MC3-R | MC4-R | MC5-R |
|---|---|---|---|
| 30% | 46% | 96% | 60% |

Ki (nM) (NDP-α-MSH) In Normal System

| MC1-R | MC3-R | MC4-R | MC5-R |
|---|---|---|---|
| 502 | 648 | 6 | 398 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | 0.38 | 215 |

In a cAMP assay for determination of functionality as to MC4-R, it was determined that the compound had in a normal system an EC₅₀ (nM) of 112, showed intrinsic activity of 0.2, and was a partial agonist as to MC4-R.

In rat model ICV feeding studies at 1 nmol dose levels, a 24 hour decrease in food intake of 4%, and decrease in body weight of 3%, was observed.

In rat model IV penile erection induction experiments at a dose of 1 μg/kg no penile erection response was observed.

### Example 21

### N—[(R)-2-[(S)-2-(4-Amino-butyl)-4-(2-naphthalen-2-yl-ethyl)-3-oxo-piperazin-1-yl]-1-(2,4-dichloro-benzyl)-2-oxo-ethyl]-4-phenoxy-benzamide

The following compound was synthesized. It was tested as described above with the results shown.

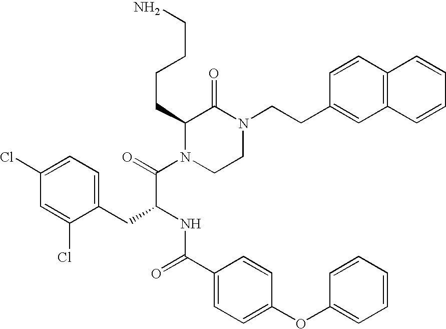

Inhibition at 1 μM (NDP-α-MSH) In Normal System

| MC1-R | MC3-R | MC4-R | MC5-R |
|---|---|---|---|
| 35% | 51% | 99% | 51% |

Ki (nM) (NDP-α-MSH) In Normal System

| MC1-R | MC3-R | MC4-R | MC5-R |
|---|---|---|---|
| 915 | 150 | 1 | 282 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | None | N/A |

In a cAMP assay for determination of functionality as to MC4-R, it was determined that the compound had no intrinsic activity.

In rat model ICV feeding studies at 1 nmol dose levels, a 24 hour decrease in food intake of 2%, and decrease in body weight of 1%, was observed.

In rat model IV penile erection induction experiments at a dose of 1 μg/kg no penile erection response was observed.

### Example 22

### (S)-1,2,3,4-Tetrahydro-isoquinoline-3-carboxylic acid {(R)-1-(2,4-dichloro-benzyl)-2-[(2S,5R)-2-(3-guanidino-propyl)-5-(naphthalen-2-yloxymethyl)-3-oxo-hexahydro-pyrrolo[1,2-a]imidazol-1-yl]-2-oxo-ethyl}-amide

The following compound was synthesized by the methods described in U.S. patent application Ser. No. 10/761,889. It was tested as described above with the results shown.

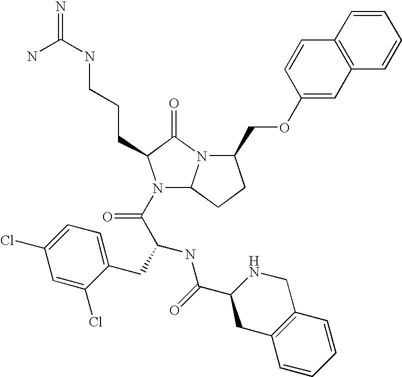

Inhibition at 1 μM (NDP-α-MSH) In Normal System

| MC1-R | MC3-R | MC4-R | MC5-R |
|---|---|---|---|
| 75% | 88% | 100% | 96% |

Ki (nM) (NDP-α-MSH) In Normal System

| MC1-R | MC3-R | MC4-R | MC5-R |
|---|---|---|---|
| 111 | 42 | 1 | 26 |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | 0.30 | 64 |

In a cAMP assay for determination of functionality as to MC4-R, it was determined that the compound had in a normal system a pA₂ value of 8.1, and was an antagonist as to MC4-R.

In rat model ICV feeding studies at 1 nmol dose levels, a 24 hour decrease in food intake of 22%, was observed.

In rat model IV penile erection induction experiments at 1 μPK, no penile erection response was observed.

### Example 23

### Heptanoyl-Thr(Bzl)-D-Phe(4-Cl)-Arg-Trp-NH₂

The peptide compound Heptanoyl-Thr(Bzl)-D-Phe(4-Cl)-Arg-Trp-NH₂ was synthesized by standard peptide synthesis methods as described in International Patent Application No. PCT/US02/22196. It was tested as described above with the results shown.

| | | | |
|---|---|---|---|
| MC1-R | MC3-R | MC4-R | MC5-R |

Inhibition at 1 μM (NDP-α-MSH) In Normal System

| | | | |
|---|---|---|---|
| 28% | 39% | 91% | 65% |

Ki (nM) (NDP-α-MSH) In Normal System

| | | | |
|---|---|---|---|
| 747 | 374 | 21 | 292 |

| Low Receptor Density System | | | |
|---|---|---|---|
| Efficacy/Intrinsic | | High Receptor Density System | |
| Activity | EC₅₀ (nM) | Efficacy/Intrinsic Activity | EC₅₀ (nM) |
| None | N/A | 0.75 | 184 |

In a cAMP assay for determination of functionality as to MC4-R, it was determined that the compound had in a normal system an EC₅₀ (nM) of >1000, and was a partial agonist as to MC4-R with an intrinsic activity of 0.4.

In rat model ICV feeding studies at 1 nmol dose levels, a 24 hour decrease in food intake of 18%, and decrease in body weight of 1%, was observed.

In rat model IV penile erection induction experiments at a dose of 1 μg/kg no penile erection response was observed.

### Example 24

### 2-naphthylacetyl-1-amino-1-cyclohexane-carbonyl-D-Phe(4-Cl)-Arg-Trp-NH₂

The peptide compound 2-naphthylacetyl-1-amino-1-cyclohexane-carbonyl-D-Phe(4-Cl)-Arg-Trp-NH₂ was synthesized by standard peptide synthesis methods as described in International Patent Application No. PCT/US02/22196. It was tested as described above with the results shown.

| | | | |
|---|---|---|---|
| MC1-R | MC3-R | MC4-R | MC5-R |

Inhibition at 1 μM (NDP-α-MSH) In Normal System

| | | | |
|---|---|---|---|
| 25% | 35% | 92% | 63% |

Ki (nM) (NDP-α-MSH) In Normal System

| | | | |
|---|---|---|---|
| 897 | 453 | 40 | 300 |

| Low Receptor Density System | | | |
|---|---|---|---|
| Efficacy/Intrinsic | | High Receptor Density System | |
| Activity | EC₅₀ (nM) | Efficacy/Intrinsic Activity | EC₅₀ (nM) |
| None | N/A | None | N/A |

In a cAMP assay for determination of functionality as to MC4-R, it was determined that the compound had in a normal system an EC₅₀ (nM) of >1000, and was a partial agonist as to MC4-R with an intrinsic activity of 0.2.

In rat model ICV feeding studies at 1 nmol dose levels, a 24 hour decrease in food intake of 21%, and decrease in body weight of 1%, was observed.

### Example 25

### N-{1(R)-(2,4-Dimethyl-benzyl)-2-[2(S)-(3-guanidino-propyl)-5(R)-methyl-4-(2-naphthalen-2-yl-ethyl)-piperazin-1-yl]-2-oxo-ethyl}-acetamide

The following compound was synthesized by the method of Scheme 5 of U.S. patent application Ser. No. 10/837,519, using 2-naphthylacetic acid as J-COOH, D-alaninol as NH₂—CH(R₅)—CH(R₄)—OH, D-alanine methyl ester as NH₂—CH(R₅—COOCH₃, and Boc-D-2,4-dimethyl-Phe-OH as Q-COOH. An acetyl group was attached to the amino group of D-2,4-dimethyl-Phe residue by the method described in Example 36 of U.S. patent application Ser. No. 10/837,519. It was tested as described above with the results shown. The mass was analyzed as 571.9 (M+H).

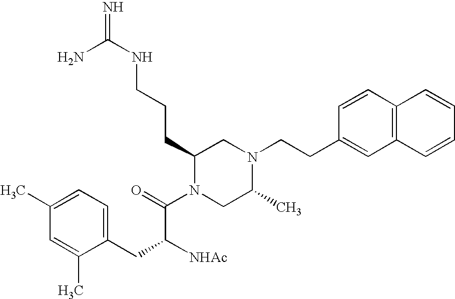

Ki (nM)

| MC1-R | MC3-R | MC4-R | MC5-R |
|---|---|---|---|
| 568 | 74 | 1 | 43 |

Inhibition at 1 μM (NDP-α-MSH) In Normal System

| MC1-R | MC3-R | MC4-R | MC5-R |
|---|---|---|---|
| 28% | 75% | 100% | 89% |

| Low Receptor Density System | | High Receptor Density System | |
|---|---|---|---|
| Efficacy/Intrinsic | | Efficacy/Intrinsic | |
| Activity | EC₅₀ (nM) | Activity | EC₅₀ (nM) |
| None | N/A | None | N/A |

In a cAMP assay using MC4-R, at 1 μM concentrations the compound of Example 26 exhibited no intrinsic activity. It tested as an MC4-R antagonist in a normal system with a pA₂ value of 8.3.

In mouse model IP feeding studies at 10 mg/kg dose levels, a 4 hour decrease in food intake of 34% was observed.

### Example 26

### N-{3-[1-[2(R)-Amino-3-(2-methyl, 4-chloro-phenyl)-propionyl]-5(R)-methyl-4-(2-naphthalen-2-yl-ethyl)-piperazin-2(S)-yl]-propyl}-guanidine

The following compound was synthesized by the methods of U.S. patent application Ser. No. 10/837,519 using 2-naphthylacetic acid as J-COOH, (R)-(−)-2-amino-1-propanol as NH₂—CH(R₅)—CH(R₄)—OH, Fmoc-Arg(Boc)₂-OH as Prt-NH—C(R₂)—COOH, D-alanine methyl ester as NH₂—CH(R₅)—COOCH₃ and Boc-D-2-methyl, 4-chloro-Phe-OH as Q-COOH. It was tested as described above with the results shown. The mass was analyzed as 549 (M+H).

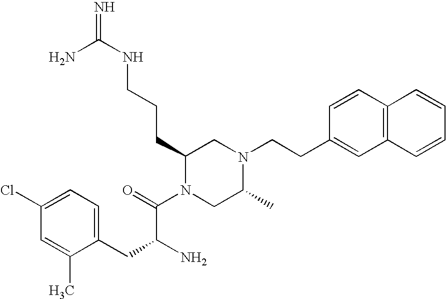

Inhibition at 1 μM (NDP-α-MSH)
In Normal System

| MC1-R | MC3-R | MC4-R | MC5-R |
|---|---|---|---|
| 6% | 74% | 100% | 77% |

Ki (nM) (NDP-α-MSH) In Normal System

| MC1-R | MC3-R | MC4-R | MC5-R |
|---|---|---|---|
| 1052 | 99 | 1 | 219 |

Low Receptor Density System

| Efficacy/Intrinsic Activity | EC₅₀ (nM) |
|---|---|
| None | N/A |

High Receptor Density System

| Efficacy/Intrinsic Activity | EC₅₀ (nM) |
|---|---|
| 0.78 | 31 |

In a cAMP assay for determination of functionality as to MC4-R, it was determined that the compound had in a normal system an EC₅₀ (nM) of 214, showed intrinsic activity of 0.3, and was a partial agonist as to MC4-R.

In mouse model IP feeding studies at 3 and 10 mg/kg dose levels, a maximum 44% and 86% decrease, respectively, was observed in food intake for a period of 4 hours.

In rat model IV penile erection induction experiments at 1 and 3 mg/kg, no penile erection were observed.

### Example 27

### N-{3-[1-[2(R)-Amino-3-(2,4-dimethyl-phenyl)-propionyl]-5(R)-methyl-4-(2-naphthalen-2-yl-ethyl)-piperazin-2(S)-yl]-propyl}-guanidine

The following compound was synthesized by the methods of U.S. patent application Ser. No. 10/837,519 using 2-naphthylacetic acid as J-COOH, (R)-(−)-2-amino-1-propanol as NH₂—CH(R₅)—CH(R₄)—OH, Fmoc-Arg(Boc)₂-OH as Prt-NH—C(R₂)—COOH, D-alanine methyl ester as NH₂—CH(R₅)—COOCH₃ and Boc-D-2,4-dimethyl-Phe-OH as Q-COOH. It was tested as described above with the results shown. The mass was analyzed as 529 (M+H).

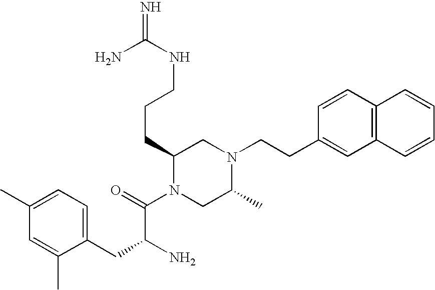

Inhibition at 1 μM (NDP-α-MSH)
In Normal System

| MC1-R | MC3-R | MC4-R | MC5-R |
|---|---|---|---|
| 9% | 62% | 100% | 59% |

Ki (nM) (NDP-α-MSH) In Normal System

| MC1-R | MC3-R | MC4-R | MC5-R |
|---|---|---|---|
| 1233 | 167 | 11 | 665 |

Low Receptor Density System

| Efficacy/Intrinsic Activity | EC₅₀ (nM) |
|---|---|
| None | N/A |

High Receptor Density System

| Efficacy/Intrinsic Activity | EC₅₀ (nM) |
|---|---|
| 0.61 | 129 |

In a cAMP assay for determination of functionality as to MC4-R, it was determined that the compound had in a normal system an EC₅₀ (nM) of 321, showed intrinsic activity of 0.2, and was a partial agonist as to MC4-R.

In mouse model IP feeding studies at 3 and 10 mg/kg dose levels, a maximum 47% and 88% decrease, respectively, was observed in food intake for a period of 4 hours.

In rat model IV penile erection induction experiments at 1 and 3 mg/kg, no penile erections were observed. In rat model ICV penile erection induction experiments at doses of 1 and 3 nmole no penile erection response was observed.

### Example 28

### N-{3-[1-[2(R)-Amino-3-(4-chloro-phenyl)-propionyl]-5(R)-methyl-4-(2-naphthalen-2-yl-ethyl)-piperazin-2(S)-yl]-propyl}-guanidine

The following compound was synthesized by the methods of U.S. patent application Ser. No. 10/837,519 using 2-naphthylacetic acid as J-COOH, (R)-(−)-2-amino-1-propanol as NH₂—CH(R₅)—CH(R₄)—OH, Fmoc-Lrg(Boc)₂-OH as Prt-NH—C(R₂)—COOH, D-alanine methyl ester as NH₂—CH(R₅)—COOCH₃ and Boc-D-4-chloro-Phe-OH as Q-COOH. It was tested as described above with the results shown. The mass was analyzed as 535 (M+H).

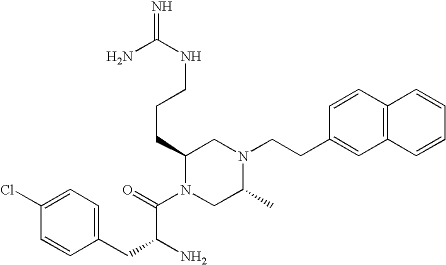

Inhibition at 1 μM (NDP-α-MSH)
In Normal System

| MC1-R | MC3-R | MC4-R | MC5-R |
|---|---|---|---|
| 64% | 70% | 97% | 61% |

Ki (nM) (NDP-α-MSH) In Normal System

| MC1-R | MC3-R | MC4-R | MC5-R |
|---|---|---|---|
| 94 | 160 | 11 | 551 |

Low Receptor Density System

| Efficacy/Intrinsic Activity | EC₅₀ (nM) |
|---|---|
| 0.29 | 106 |

High Receptor Density System

| Efficacy/Intrinsic Activity | EC₅₀ (nM) |
|---|---|
| 0.75 | 24 |

In a cAMP assay for determination of functionality as to MC4-R, it was determined that the compound had in a normal system an EC₅₀ (nM) of 252, showed intrinsic activity of 0.8, and was an agonist as to MC4-R.

In mouse model IP feeding studies at 3 mg/kg dose levels, a maximum 46% decrease was observed in food intake for a period of 4 hours.

In rat model IV penile erection induction experiments at doses ranging 0.003 to 3 mg/kg, no penile erections were observed.

### Example 29

### N-{3-[1-[2(R)-Amino-3-(4-chloro-phenyl)-propionyl]-5(R)-methyl-4-(2-naphthalen-2-yl-ethyl)-piperazin-2(S)-yl]-propyl}-guanidine

The following compound was synthesized by the method of Scheme 7 of U.S. patent application Ser. No. 10/837,519 using 2-naphthyl acetic acid as J-COOH, L-Orn(Boc) methyl ester as NH₂—CH(R₂)—COOCH₃, and Boc-D-2,4-di-methyl-Phe-OH as Q-COOH. It was tested as described above with the results shown. The mass was analyzed as 543 (M+H).

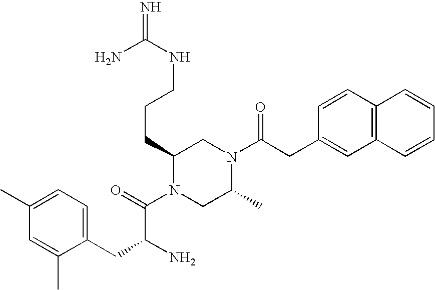

Inhibition at 1 μM (NDP-α-MSH)
In Normal System

| MC1-R | MC3-R | MC4-R | MC5-R |
|---|---|---|---|
| 0% | 25% | 85% | 7% |

Ki (nM) (NDP-α-MSH) In Normal System

| MC1-R | MC3-R | MC4-R | MC5-R |
|---|---|---|---|
| 431 | 526 | 6 | 1536 |

Low Receptor Density System

| Efficacy/Intrinsic Activity | EC₅₀ (nM) |
|---|---|
| 0.25 | 45 |

High Receptor Density System

| Efficacy/Intrinsic Activity | EC₅₀ (nM) |
|---|---|
| 0.77 | 34 |

In a cAMP assay for determination of functionality as to MC4-R, it was determined that the compound had in a normal system an EC₅₀ (nM) of 46, showed intrinsic activity of 0.8, and was an agonist as to MC4-R.

In mouse model IP feeding studies at 3 and 10 mg/kg dose levels, a 3% and 26% decrease, respectively, was observed in food intake for a period of 20 hours.

In rat model IV penile erection induction experiments at doses ranging 0.0003 to 0.03 mg/kg, it exhibited a maximum of 0.5 mean PEs per rat with no more than 50% animals responding.

### Example 30

### N-{(R)-1-(2,4 dimethyl-benzyl)-2-[(2S,5R)-2-(3-guanidino-propyl)-5-methyl-4-(2-naphthalen-2-yl-acetyl)-piperazin-1-yl]-2-oxo-ethyl}-acetamide

The following compound was synthesized by the method used for Example 29, with an acetyl group introduced at the amino group of D-2,4-dimethyl-Phe by reaction of the compound of Example 29 with Ac-OSu in DMF. It was tested as described above with the results shown. The mass was analyzed as 585 (M+H).

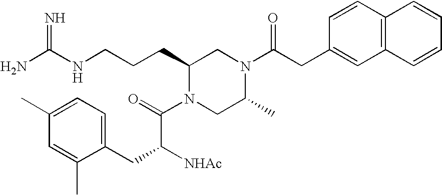

Ki (nM) (NDP-α-MSH) In Normal System

| MC1-R | MC3-R | MC4-R | MC5-R |
|---|---|---|---|
| 1883 | 232 | 11 | 250 |

Low Receptor Density System

| Efficacy/Intrinsic Activity | EC₅₀ (nM) |
|---|---|
| None | N/A |

High Receptor Density System

| Efficacy/Intrinsic Activity | EC₅₀ (nM) |
|---|---|
| 0.18 | 51 |

In a cAMP assay for determination of functionality as to MC4-R, it was determined that the compound had in a normal system an EC₅₀ (nM) of 9, showed intrinsic activity of 0.4, and was a partial agonist as to MC4-R.

In mouse model IP feeding studies at 3 mg/kg dose levels, a maximum 8% decrease was observed in food intake for a period of 4 hours.

In rat model IV penile erection induction experiments at doses ranging 0.0003 to 0.03 mg/kg, it exhibited no penile activity response.

### Example 31

### N-{(R)-1-(2-methyl, 4-chloro-benzyl)-2-[(2S,5R)-2-(3-guanidino-propyl)-5-methyl-4-(2-naphthalen-2-yl-acetyl)-piperazin-1-yl]-2-oxo-ethyl}-acetamide

The following compound was synthesized by the method of Scheme 7 of U.S. patent application Ser. No. 10/837,519 using 2-naphthyl acetic acid as J-COOH, L-Orn(Boc) methyl ester as NH₂—CH(R₂)—COOCH₃, and Boc-D-4-chloro-2-methyl-Phe-OH as Q-COOH with an acetyl group introduced at the amino group of the D-4-chloro-2-methyl-Phe residue by reaction of the compound with Ac-OSu in DMF. It was tested as described above with the results shown. The mass was analyzed as 605 (M+H).

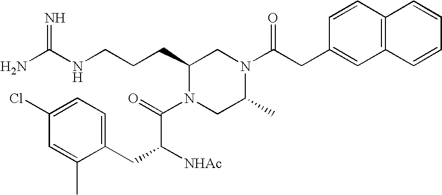

Ki (nM) (NDP-α-MSH) In Normal System

| MC1-R | MC3-R | MC4-R | MC5-R |
|---|---|---|---|
| 1388 | 136 | 5 | 234 |

Low Receptor Density System

| Efficacy/Intrinsic Activity | EC₅₀ (nM) |
|---|---|
| None | N/A |

High Receptor Density System

| Efficacy/Intrinsic Activity | EC₅₀ (nM) |
|---|---|
| 0.27 | 39 |

In a cAMP assay for determination of functionality as to MC4-R, it was determined that the compound had in a normal system an EC₅₀ (nM) of 3, showed intrinsic activity of 0.6, and was a partial agonist as to MC4-R.

In mouse model IP feeding studies at 10 mg/kg dose levels, a maximum 7% decrease, was observed in food intake for a period of 4 hours.

In rat model IV penile erection induction experiments at doses ranging 0.0003 to 0.03 mg/kg, it exhibited no penile activity response.

### Example 32

### N-{(R)-2-[(2S,5R)-2-(3-guanidino-propyl)-5-methyl-4-(2-naphthalen-2-yl-acetyl)-piperazin-1-yl]-1-naphthalen-2-ylmethyl-2-oxo-ethyl}-acetamide

The following compound was synthesized by the method of Scheme 6 of U.S. patent application Ser. No. 10/837,519 using 2-naphthylacetic acid as J-COOH, D-alanine methyl ester as NH₂—CH(R₅)—COOCH₃, and Boc-D-2-Nal-OH as Q-COOH with an acetyl group attached to the amino group of D-2-Nal by reaction of the compound with Ac-OSu in DMF. It was tested as described above with the results shown. The mass was analyzed as 605 (M+H).

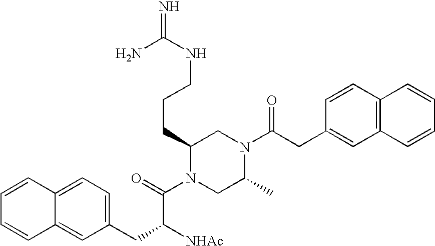

Inhibition at 1 μM (NDP-α-MSH)
In Normal System

| MC1-R | MC3-R | MC4-R | MC5-R |
|---|---|---|---|
| 51% | 38% | 97% | 44% |

Ki (nM) (NDP-α-MSH) In Normal System

| MC1-R | MC3-R | MC4-R | MC5-R |
|---|---|---|---|
| 406 | 634 | 7 | 826 |

Low Receptor Density System

| Efficacy/Intrinsic Activity | EC₅₀ (nM) |
|---|---|
| None | N/A |

High Receptor Density System

| Efficacy/Intrinsic Activity | EC₅₀ (nM) |
|---|---|
| None | N/A |

In a cAMP assay for determination of functionality as to MC4-R, it was determined that the compound had in a normal system an EC₅₀ (nM) of 7, showed intrinsic activity of 0.3, and was a partial agonist as to MC4-R.

In mouse model IP feeding studies at 10 mg/kg dose levels, a maximum 34% decrease was observed in food intake for a period of 4 hours.

In rat model ICV penile erection induction experiments at doses of 0.1 and 1 nmole, it exhibited no penile erection response.

### Example 33

### N-{3-[(2S)-1-[(R)-2-Amino-3-(naphthlene-2-yl)-propionyl]-5(R)-methyl-4-(2-naphthalen-2-yl-acetyl)-piperazin-2-yl]-propyl}-guanidine

The following compound was synthesized by the method of Scheme 6 of U.S. patent application Ser. No. 10/837,519 using 2-naphthylacetic acid as J-COOH, D-alanine methyl ester as NH₂—CH(R₅)—COOCH₃, and Boc-D-2-Nal-OH as Q-COOH. It was tested as described above with the results shown. The mass was analyzed as 605 (M+H).

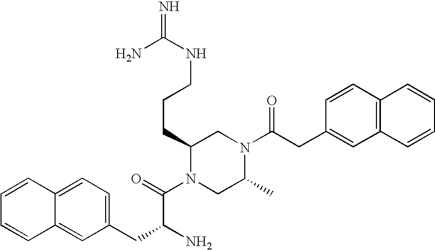

Inhibition at 1 μM (NDP-α-MSH)
In Normal System

| MC1-R | MC3-R | MC4-R | MC5-R |
|---|---|---|---|
| 43% | 40% | 89% | 50% |

Ki (nM) (NDP-α-MSH) In Normal System

| MC1-R | MC3-R | MC4-R | MC5-R |
|---|---|---|---|
| 227 | 596 | 19 | 548 |

Low Receptor Density System

| Efficacy/Intrinsic Activity | EC₅₀ (nM) |
|---|---|
| None | N/A |

High Receptor Density System

| Efficacy/Intrinsic Activity | EC₅₀ (nM) |
|---|---|
| 0.73 | 50 |

In a cAMP assay for determination of functionality as to MC4-R, it was determined that the compound had in a normal system an EC₅₀ (nM) of 42, showed intrinsic activity of 1, and was an agonist as to MC4-R.

In mouse model IP feeding studies at 10 mg/kg dose levels, a maximum 25% decrease was observed in food intake for a period of 4 hours.

In rat model IV penile erection induction experiments at doses of 0.0003 to 0.03 mg/kg, it exhibited no penile erection response.

### Example 34

### Ac-Nle-cyclo(Asp-His-D-Nal 2-Arg-Nal 2-Lys)-OH

The following peptide compound was synthesized by standard peptide synthesis methods. It was tested as described above with the results shown.

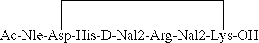

Inhibition at 1 μM (NDP-α-MSH)
In Normal System

| MC1-R | MC3-R | MC4-R | MC5-R |
|---|---|---|---|
| 102% | 98% | 100% | 100% |

Ki (nM) (NDP-α-MSH) In Normal System

| MC1-R | MC3-R | MC4-R | MC5-R |
|---|---|---|---|
| 0.2 | 0.3 | 0.02 | 0.2 |

Low Receptor Density System

| Efficacy/Intrinsic Activity | EC₅₀ (nM) |
|---|---|
| None | N/A |

High Receptor Density System

| Efficacy/Intrinsic Activity | EC₅₀ (nM) |
|---|---|
| None | N/A |

In a cAMP assay for determination of functionality as to MC4-R, it was determined that the compound was inactive at 1 nM dose. It tested as an MC4-R antagonist in a normal system with a pA₂ value of 8.1. In separate experiments, human MC1-R was used to determine the Ki (nM) (NDP-α-MSH) in a normal system, with a Ki of 0.25 nM.

In rat model ICV feeding studies at 1 nmol dose levels, a 24 hour increase in food intake of 12 g, and increase in body weight of 16 g, was observed.

In an ICV rat model of PE efficacy the compound was inactive when administered at a dose of 3 nanomole.

### Example 35

### Ac-cyclo(Asp-Trp-D-Nal 2-Arg-Nal 2-Lys)-NH—CH₃

The following peptide compound was synthesized by standard peptide synthesis methods. It was tested as described above with the results shown.

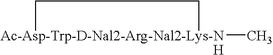

Inhibition at 1 μM (NDP-α-MSH)
In Normal System

| MC1-R | MC3-R | MC4-R | MC5-R |
|---|---|---|---|
| 67% | 99% | 100% | 98% |

Ki (nM) (NDP-α-MSH) In Normal System

| MC1-R | MC3-R | MC4-R | MC5-R |
|---|---|---|---|
| 643 | 3 | 0.06 | 6 |

Low Receptor Density System

| Efficacy/Intrinsic Activity | EC₅₀ (nM) |
|---|---|
| None | N/A |

High Receptor Density System

| Efficacy/Intrinsic Activity | EC₅₀ (nM) |
|---|---|
| None | N/A |

In a cAMP assay for determination of functionality as to MC4-R, it was determined that the compound was inactive at 1 nM dose.

In rat model ICV feeding studies at 1 nmol dose levels, a 24 hour increase in food intake of 6 g, and increase in body weight of 6 g, was observed. In mouse model IP feeding studies at 1 mg/kg dose levels, a maximal 4% decrease was observed in food intake for a period of 4 hours.

In an ICV rat model of PE efficacy, the compound was inactive when administered at a dose of 3 nM.

### Example 36

### Ac-Nle-cyclo(-Asp-Trp-D-Phe-Arg-Nal 1-Lys)-N(CH₃)₂

The following peptide compound was synthesized by standard peptide synthesis methods. It was tested as described above with the results shown.

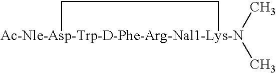

Inhibition at 1 μM (NDP-α-MSH)
In Normal System

| MC1-R | MC3-R | MC4-R | MC5-R |
|---|---|---|---|
| 71% | 83% | 99% | 92% |

Ki (nM) (NDP-α-MSH) In Normal System

| MC1-R | MC3-R | MC4-R | MC5-R |
|---|---|---|---|
| 12 | 231 | 38 | 31 |

Low Receptor Density System

| Efficacy/Intrinsic Activity | EC₅₀ (nM) |
|---|---|
| None | N/A |

High Receptor Density System

| Efficacy/Intrinsic Activity | EC₅₀ (nM) |
|---|---|
| 0.98 | 4 |

In a cAMP assay for determination of functionality as to MC4-R, it was determined that the compound had in a normal system an EC₅₀ (nM) of 272, showed intrinsic activity of 0.8, and was an agonist as to MC4-R. In separate experiments, human MC1-R was used to determine the Ki (nM) (NDP-α-MSH) in a normal system, with a Ki of 39 nM.

### Example 37

### Ac-Nle-cyclo(-Asp-His-D-Phe-Arg-Nal 1-Lys)-N(CH₃)₂

The following peptide compound was synthesized by standard peptide synthesis methods. It was tested as described above with the results shown.

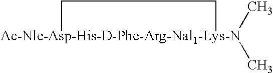

Inhibition at 1 μM (NDP-α-MSH)
In Normal System

| MC1-R | MC3-R | MC4-R | MC5-R |
|---|---|---|---|
| 82% | 92% | 100% | 92% |

Ki (nM) (NDP-α-MSH) In Normal System

| MC1-R | MC3-R | MC4-R | MC5-R |
|---|---|---|---|
| 0.39 | 111 | 5 | 50 |

Low Receptor Density System

| Efficacy/Intrinsic Activity | EC₅₀ (nM) |
|---|---|
| 0.69 | 2.1 |

High Receptor Density System

| Efficacy/Intrinsic Activity | EC₅₀ (nM) |
|---|---|
| 0.88 | 0.6 |

In a cAMP assay for determination of functionality as to MC4-R, it was determined that the compound had in a normal system an EC₅₀ (nM) of 44, showed intrinsic activity of 0.7, and was an agonist as to MC4-R. In separate experiments, human MC1-R was used to determine the Ki (nM) (NDP-α-MSH) in a normal system, with a Ki of 0.08 nM.

### Example 38

### Ac-Nle-cyclo(-Asp-Trp-D-Phe-Arg-Nal 1-Lys)-NH—CH₂—CH₃

The following peptide compound was synthesized by standard peptide synthesis methods. It was tested as described above with the results shown.

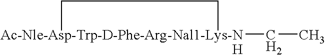

Inhibition at 1 μM (NDP-α-MSH)
In Normal System

| MC1-R | MC3-R | MC4-R | MC5-R |
|---|---|---|---|
| 98% | 92% | 100% | 92% |

Ki (nM) (NDP-α-MSH) In Normal System

| MC1-R | MC3-R | MC4-R | MC5-R |
|---|---|---|---|
| 4.2 | 77 | 9 | 47 |

Low Receptor Density System

| Efficacy/Intrinsic Activity | EC₅₀ (nM) |
|---|---|
| None | N/A |

High Receptor Density System

| Efficacy/Intrinsic Activity | EC₅₀ (nM) |
|---|---|
| 1.0 | 10.0 |

In a cAMP assay for determination of functionality as to MC4-R, it was determined that the compound had in a normal system an EC₅₀ (nM) of 120, showed intrinsic activity of 0.6, and was an agonist as to MC4-R. In separate experiments, human MC1-R was used to determine the Ki (nM) (NDP-α-MSH) in a normal system, with a Ki of 30 nM.

### Example 39

### Ac-Nle-cyclo(-Asp-His-D-Phe-Arg-Nal 1-Lys)-NH—CH₂—CH₃

The following peptide compound was synthesized by standard peptide synthesis methods. It was tested as described above with the results shown.

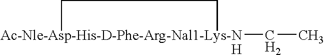

Inhibition at 1 μM (NDP-α-MSH)
In Normal System

| MC1-R | MC3-R | MC4-R | MC5-R |
|---|---|---|---|
| 100% | 93% | 98% | 93% |

Ki (nM) (NDP-α-MSH) In Normal System

| MC1-R | MC3-R | MC4-R | MC5-R |
|---|---|---|---|
| 0.07 | 28 | 2 | 63 |

Low Receptor Density System

| Efficacy/Intrinsic Activity | EC₅₀ (nM) |
|---|---|
| 0.77 | 10 |

High Receptor Density System

| Efficacy/Intrinsic Activity | EC₅₀ (nM) |
|---|---|
| 1.0 | 1.1 |

In a cAMP assay for determination of functionality as to MC4-R, it was determined that the compound had in a normal system an EC₅₀ (nM) of 0.2, showed intrinsic activity of 0.6, and was a partial agonist as to MC4-R. In separate experiments, human MC1-R was used to determine the Ki (nM) (NDP-α-MSH) in a normal system, with a Ki of 0.04 nM.

### Example 40

### Ac-cyclo(-Asp-His-D-Phe-Arg-Trp-Lys)-NH—CH₂—CH₃

The following peptide compound was synthesized by standard peptide synthesis methods. It was tested as described above with the results shown.

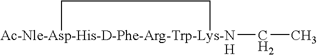

Inhibition at 1 μM (NDP-α-MSH)
In Normal System

| MC1-R | MC3-R | MC4-R | MC5-R |
|---|---|---|---|
| 96% | 65% | 96% | 16% |

Ki (nM) (NDP-α-MSH) In Normal System

| MC1-R | MC3-R | MC4-R | MC5-R |
|---|---|---|---|
| 5 | 84 | 6 | >1,000 |

Low Receptor Density System

| Efficacy/Intrinsic Activity | EC₅₀ (nM) |
|---|---|
| 1.0 | 3.1 |

High Receptor Density System

| Efficacy/Intrinsic Activity | EC₅₀ (nM) |
|---|---|
| 1.0 | 0.5 |

In a cAMP assay for determination of functionality as to MC4-R, it was determined that the compound had in a normal system an EC₅₀ (nM) of 3, showed intrinsic activity of 0.6, and was a partial agonist as to MC4-R. In separate experiments, human MC1-R was used to determine the Ki (nM) (NDP-α-MSH) in a normal system, with a Ki of 0.52 nM.

In rat model IV penile erection induction experiments, the compound was active and showed peak PE activity when administered at a dose of 0.3 mg/kg, with 2.5 mean penile erections per rat observed with 83% of the animals responding.

The preceding examples can be repeated with similar success by substituting the generically or specifically described reagents, reactions and/or operating conditions of this invention for those used in the preceding examples. In particular, and without limiting the generality of the foregoing, it is possible to use other methods of transformation, transfection or expressing differential densities or levels of melanocortin receptors, other sources of melanocortin receptors, or cell types and systems, other assay systems, other methods of determining functional activity, and the like.

Although the invention has been described in detail with particular reference to these preferred embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference as if set forth in full.

What is claimed is:

1. A method of selecting a compound as a candidate for attenuating food intake which compound at least substantially does not induce or initiate a sexual response, the method comprising the steps of:
providing a low melanocortin 4 receptor (MC4-R) density system;
providing a high density MC4-R system;
determining the functional activity of the compound in the low density MC4-R system;
determining the functional activity of the compound in the high density MC4-R system; and
selecting the compound as a candidate if the compound is functionally inactive in the low MCR-4 density system and is functionally active in the high density MC4-R system.

2. The method of claim 1, wherein determining the functional activity of the compound in the low density and high density MC4-R system comprises determining activation of melanocortin 4 receptors in each of the low density and high density MC4-R systems.

3. The method of claim 1, wherein determining the functional activity of the compound in the low density and high density MC4-R system comprises an adenylyl cyclase activity assay.

4. The method of claim 1, wherein the low density MC4-R system is a low human MC4-R (hMC4-R) density system and the high density MC4-R system is a high hMC4-R density system.

5. The method of claim 1, wherein the low density MC4-R system has a melanocortin receptor density determined by receptor binding saturation that is at least ten times lower than the melanocortin receptor density in the high density MC4-R system.

6. The method of claim 5, wherein the low density MC4-R system has a melanocortin receptor density determined by receptor binding saturation that is at least fifty times lower than the melanocortin receptor density in the high density MC4-R system.

7. The method of claim 5, wherein the low density MC4-R system has a melanocortin receptor density determined by receptor binding saturation that is at least one hundred times lower than the melanocortin receptor density in the high density MC4-R system.

8. The method of claim 1, wherein both the low density MC4-R system and the high density MC4-R system comprise mammalian cells transformed or transfected with a gene encoding MC4-R, which gene is expressible in the cells.

9. The method of claim 8, wherein the gene encoding for MC4-R comprises a gene encoding for hMC4-R.

10. The method of claim 8, wherein the gene encoding for MC4-R is under the control of a gene expression variable regulatory system.

11. The method of claim 10, wherein the gene expression variable regulatory system comprises a tetracycline-regulated mammalian expression system.

12. The method of claim 5, wherein the low density MC4-R system has a receptor density expressed as a Bₘₐₓ receptor binding saturation value using NDP-α-MSH of between approximately 80 and 20 fmol/mg.

13. The method of claim 5, wherein the high density MC4-R system has a receptor density expressed as a Bₘₐₓ receptor binding saturation value using NDP-α-MSH of at least about 4,000 fmol/mg.

14. The method of claim 8, wherein the mammalian cells comprise HEK-293 cells.

15. The method of claim 1, wherein the compound is functionally inactive in the low MCR-4 density system where the compound has no measurable intrinsic activity.

16. The method of claim 1, wherein the compound is functionally inactive in the low MCR-4 density system and has an intrinsic activity of less than about 0.1 (10%), based on maximal stimulation of adenylyl cyclase achievable by the compound in the low hMC4-R density system wherein the maximal stimulation achieved by α-MSH or NDP-α-MSH is designated as an intrinsic activity of 1.0 (100%).

17. The method of claim 1, wherein the compound is functionally active in the high density MC4-R system and has an intrinsic activity of more than about 0.1 (10%), based on maximal stimulation of adenylyl cyclase achievable by the compound in the high hMC4-R density system wherein the maximal stimulation achieved by α-MSH or NDP-α-MSH is designated as an intrinsic activity of 1.0 (100%).

18. The method of claim 17, wherein the compound is functionally active in the high density MC4-R system and has an intrinsic activity of more than about 0.2 (20%), based on maximal stimulation of adenylyl cyclase achievable by the compound in the high hMC4-R density system wherein the maximal stimulation achieved by α-MSH or NDP-α-MSH is designated as an intrinsic activity of 1.0 (100%).

19. The method of claim 17, wherein the compound is functionally active in the high density MC4-R system and has an intrinsic activity of more than about 0.3 (30%), based on maximal stimulation of adenylyl cyclase achievable by the compound in the high hMC4-R density system wherein the maximal stimulation achieved by α-MSH or NDP-α-MSH is designated as an intrinsic activity of 1.0 (100%).

20. The method of claim 17, wherein the compound is functionally active in the high density MC4-R system and has an intrinsic activity of more than about 0.5 (50%), based on maximal stimulation of adenylyl cyclase achievable by the compound in the high hMC4-R density system wherein the maximal stimulation achieved by α-MSH or NDP-α-MSH is designated as an intrinsic activity of 1.0 (100%).

21. The method of claim 17, wherein the compound is functionally active in the high density MC4-R system and has an intrinsic activity of more than about 0.7 (70%), based on maximal stimulation of adenylyl cyclase achievable by the compound in the high hMC4-R density system wherein the maximal stimulation achieved by α-MSH or NDP-α-MSH is designated as an intrinsic activity of 1.0 (100%).

22. A method of selecting a compound as a candidate for treating sexual dysfunction, the method comprising the steps of:
providing a low density MC4-R system;
providing a high density MC4-R system;
determining the functional activity of the compound in the low density MC4-R system;
determining the functional activity of the compound in the high density MC4-R system; and
selecting the compound as a candidate if the compound is functionally active in both the low MCR-4 density system and high density MC4-R system.

23. The method of claim 22, wherein determining the functional activity of the compound in the low density and high density MC4-R system comprises determining activation of melanocortin 4 receptors in each of the low density and high density MC4-R systems.

24. The method of claim 22, wherein determining the functional activity of the compound in the low density and high density MC4-R system comprises an adenylyl cyclase activity assay.

25. The method of claim 22, further comprising the step of: determining the functional status of the compound in a transformed MC4-R system not under the control of a gene expression variable regulatory system, and wherein selecting the compound as a candidate for treating sexual dysfunction further requires that the compound is an agonist in the transformed MC4-R system not under the control of a gene expression variable regulatory system.

26. The method of claim 25, wherein the compound is an agonist in the transformed MC4-R system and has an intrinsic activity of more than about 0.7 (70%), based on maximal stimulation of adenylyl cyclase achievable by the compound in the transformed hMC4-R system wherein the maximal stimulation achieved by α-MSH or NDP-α-MSH is designated as an intrinsic activity of 1.0 (100%).

27. The method of claim 22, wherein the low density MC4-R system is a low human MC4-R (hMC4-R) density system and the high density MC4-R system is a high hMC4-R density system.

28. The method of claim 22, wherein the low density MC4-R system has a melanocortin receptor density determined by receptor binding saturation that is at least ten times lower than the melanocortin receptor density in the high density MC4-R system.

29. The method of claim 28 wherein the low density MC4-R system has a melanocortin receptor density determined by receptor binding saturation that is at least fifty times lower than the melanocortin receptor density in the high density MC4-R system.

30. The method of claim 28, wherein the low density MC4-R system has a melanocortin receptor density determined by receptor binding saturation that is at least one hundred times lower that the melanocortin receptor density in the high density MC4-R system.

31. The method of claim 22, wherein both the low density MC4-R system and the high density MC4-R system comprise mammalian cells transformed or transfected with a gene encoding MC4-R, which gene is expressible in the cells.

32. The method of claim 31, wherein the gene encoding for MC4-R comprises a gene encoding for hMC4-R.

33. The method of claim 31, wherein the gene encoding for MC4-R is under the control of a gene expression variable regulatory system.

34. The method of claim 33, wherein the gene expression variable regulatory system comprises a tetracycline-regulated mammalian expression system that is regulatable by tetracycline or a related regulator.

35. A method of selecting a melanotropin receptor binding compound as a candidate for attenuating food intake which compound at least substantially does not induce or initiate a sexual response, the method comprising the steps of:
providing a low density MC4-R system;
providing a high density MC4-R system;
determining the functional activity of the compound in the low density MC4-R system;
determining the functional activity of the compound in the high density MC4-R system; and
selecting the compound as a candidate if the compound is functionally inactive in both the low MCR-4 density system and the high density MC4-R system.

36. The method of claim 35, further comprising the step of:
providing a competition inhibition assay for competitive binding with a MC4-R agonist as to MC4-R,
wherein selecting the compound as a candidate for attenuating food intake further requires that the compound inhibits at least about 90% of the binding of the MC4-R agonist to MC4-R.

37. The method of claim 36, where the MC4-R agonist is α-MSH.

38. The method of claim 37, further comprising the step of:
providing an assay for determining the Ki (nM) of the compound as to α-MSH binding to MC4-R,
wherein selecting the compound as a candidate for attenuating food intake further requires that the compound has a Ki (nM) of less than about 100.

39. The method of claim 38, wherein the compound has a Ki (nM) of less than about 10.

40. The method of claim 38, wherein the compound has a Ki (nM) of less than about 1.

41. The method of claim 35, wherein the low density MC4-R system is a low human MC4-R (hMC4-R) density system and the high density MC4-R system is a high hMC4-R density system.

42. The method of claim 35, wherein the compound is functionally inactive in both the low MCR-4 density system and the high density MC4-R system wherein the compound has an intrinsic activity of less than about 0.1 (10%), based on maximal stimulation of adenylyl cyclase achievable by the compound in each of the low and high MC4-R density system wherein the maximal stimulation achieved by α-MSH or NDP-α-MSH is designated as an intrinsic activity of 1.0 (100%).

43. A pharmaceutical composition for attenuating food intake without substantially inducing a sexual response, comprising:
an effective amount of a melanocortin-4 receptor (MC4-R) specific compound which is functionally inactive at a concentration of about 1000 nM or less in an adenylyl cyclase activity assay at a low density MC4-R and which is functionally active at a concentration of about 1000 nM or less in an adenylyl cyclase activity assay at a high density MC4-R; and
one or more of a carrier, an excipient and an adjunct ingredient.

44. A pharmaceutical composition for attenuating food intake without substantially inducing a sexual response, comprising:
an effective amount of a melanocortin-4 receptor (MC4-R) specific compound which is functionally inactive at a concentration of about 1000 nM or less in an adenylyl cyclase expression assay at a low density MC4-R and at a high density MC4-R; and
one or more of a carrier, an excipients and an adjunct ingredient.

45. A pharmaceutical composition for treatment of sexual dysfunction, comprising:
an effective amount of a melanocortin-4 receptor (MC4-R) specific compound which is functionally active at a concentration of about 1000 nM or less in an adenylyl cyclase activity assay at both a low density MC4-R and at a high density MC4-R; and
one or more of a carrier, an excipient and an adjunct ingredient.
